(19) United States
(12) Patent Application Publication
(10) Pub. No.: US 2025/0072999 A1
(43) Pub. Date: Mar. 6, 2025

(54) ORTHODONTIC TREATMENT PLANNING BASED ON REINFORCEMENT LEARNING

(71) Applicant: Align Technology, Inc., San Jose, CA (US)

(72) Inventors: John Y. Morton, San Jose, CA (US); Christopher E. Cramer, Durham, NC (US); Chad Clayton Brown, Cary, NC (US); Avi Kopelman, Palo Alto, CA (US)

(73) Assignee: Align Technology, Inc., San Jose, CA (US)

(21) Appl. No.: 18/818,404

(22) Filed: Aug. 28, 2024

Related U.S. Application Data
(60) Provisional application No. 63/536,022, filed on Aug. 31, 2023.

(51) Int. Cl. A61C 7/00; A61C 7/12; G16H 20/40; G16H 50/70

(52) U.S. Cl. CPC A61C 7/002; A61C 7/12; G16H 20/40; G16H 50/70

(57) ABSTRACT

A method comprises receiving, by a processing device, clinical data of a first state of one or more dental arches of a patient. The method further comprises determining, by the processing device, one or more stages of an orthodontic treatment plan for correcting the one or more dental arches based on processing of the clinical data. Determining a stage of the orthodontic treatment plan comprises determining one or more actions to be performed with respect to the one or more dental arches and determining a target state of the one or more dental arches that is predicted to result at least in part from the one or more actions.

### RELATED APPLICATIONS

This patent application claims the benefit under 35 U.S.C. § 119 (e) of U.S. Provisional Application No. 63/536,022, filed Aug. 31, 2023, which is incorporated by reference herein.

### TECHNICAL FIELD

Embodiments of the present invention relate to the field of dentistry and, in particular, to a system and method for automated orthodontic treatment planning.

### BACKGROUND

Orthodontic treatment may be performed using a series of aligners and/or other orthodontic appliances. Generally, an orthodontist determines an orthodontic treatment plan and orders the series of aligners. A patient may wear a series of aligners in a predetermined sequence to adjust the patient's teeth from a starting configuration to a final target configuration. A doctor generally makes one or more clinical decisions associated with the orthodontic treatment plan, which has a large impact on the success of failure of the orthodontic treatment plan. Additionally, in many instances the teeth do not move as planned in the orthodontic treatment plan. However, it can be difficult for the doctor to identify which teeth might be moving according to plan and which teeth might be lagging or not moving according to plan. Additionally, it may also be difficult for the doctor to identify other deviations from the orthodontic treatment plan during treatment, to determine why teeth are not moving, and to determine how to remedy the deviation from the treatment plan. It can also be challenging for a doctor to determine an optimal orthodontic treatment plan for a patient, to determine what clinical actions to perform for the patient at one or more stages of treatment, to determine an optimal number of stages of treatment, and so on.

### SUMMARY

In a 1^{st} aspect of the disclosure, a method comprises: receiving, by a processing device, clinical data of a first state of a dentition (e.g., one or more dental arches) of a patient; and determining, by the processing device (with or without user input), one or more stages of an orthodontic treatment plan for correcting the dentition (e.g., one or more dental arches) based on processing of the clinical data, wherein determining a stage of the orthodontic treatment plan comprises: determining one or more actions to be performed with respect to the dentition (e.g., one or more dental arches); and determining a target state of the dentition (e.g., one or more dental arches) that is predicted to result at least in part from the one or more actions.

A 2^{nd} aspect of the disclosure may further extend the 1^{st} aspect of the disclosure. In the 2^{nd} aspect of the disclosure, the method further comprises: determining the orthodontic treatment plan comprising the one or more stages; determining one or more additional orthodontic treatment plans; outputting the orthodontic treatment plan and the one or more additional orthodontic treatment plans to a display; receiving a selection of the orthodontic treatment plan; and implementing the selected orthodontic treatment plan.

A 3rd aspect of the disclosure may further extend the 2^{nd} aspect of the disclosure. In the 3^{rd} aspect of the disclosure, the method further comprises: determining, for the orthodontic treatment plan, a first score associated with predicted accomplishment of one or more target conditions by the orthodontic treatment plan; and determining, for each additional orthodontic treatment plan of the one or more additional orthodontic treatment plans, an additional score associated with accomplishment of the one or more target conditions by the additional orthodontic treatment plan.

A 4^{th} aspect of the disclosure may further extend the 3^{rd} aspect of the disclosure. In the 4^{th} aspect of the disclosure, the first score and the additional scores of the one or more additional orthodontic treatment plans are output to the display.

A 5^{th} aspect of the disclosure may further extend and of the 1^{st} through 4^{th} aspects of the disclosure. In the 5^{th} aspect of the disclosure, the processing of the clinical data is performed using a trained machine learning model, wherein the trained machine learning model outputs the one or more actions and the target state of the one or more dental arches.

A 6^{th} aspect of the disclosure may further extend the 5^{th} aspect of the disclosure. In the 6^{th} aspect of the disclosure, the first state of the one or more dental arches is an intermediate state achieved by a previously determined orthodontic treatment plan, the method further comprising: receiving information associated with the previously determined orthodontic treatment plan; determining a cost value or reward value based on a degree of similarity between the first state of the one or more dental arches and a predicted state of the one or more dental arches from the previously determined orthodontic treatment plan; and updating a training of the trained machine learning model based on the cost value or reward value, wherein the processing of the clinical data using the trained machine learning model is performed after updating the training of the trained machine learning model.

A 7^{th} aspect of the disclosure may further extend any of the 1^{st} through 6^{th} aspects of the disclosure. In the 7^{th} aspect of the disclosure, the receiving of the clinical data and the determining of the one or more stages of the orthodontic treatment plan is performed prior to beginning orthodontic treatment of the one or more dental arches, the method further comprising: receiving, by the processing device, new clinical data of a new state of the one or more dental arches during an intermediate stage of the orthodontic treatment plan; and determining, by the processing device with or without user input, one or more updated stages of the orthodontic treatment plan based on processing of the new clinical data.

An 8^{th} aspect of the disclosure may further extend the 7^{th} aspect of the disclosure. In the 8^{th} aspect of the disclosure, the one or more updated stages comprise a final stage having a new target state of the one or more dental arches.

A 9^{th} aspect of the disclosure may further extend the 7^{th} or 8^{th} aspect of the disclosure. In the 9^{th} aspect of the disclosure, the method further comprises adding one or more new intermediate stages to the orthodontic treatment plan in view of the new clinical data.

A 10^{th} aspect of the disclosure may further extend any of the 1^{st} through 9^{th} aspects of the disclosure. In the 10^{th} aspect of the disclosure, the clinical data comprises at least one a color two-dimensional (2D) image of the one or more dental arches, a three-dimensional (3D) model of each of the one or more dental arches, an intraoral scan of the one or more dental arches, or an x-ray image (e.g., a radiograph) of the one or more dental arches.

An 11^{th} aspect of the disclosure may further extend any of the 1^{st} through 10^{th} aspects of the disclosure. In the 11^{th} aspect of the disclosure, the one or more actions comprise at least one of widening the one or more dental arches, adding one or more attachments to the one or more dental arches, extracting one or more teeth from the one or more dental arches, performing interproximal reduction for one or more teeth of the one or more dental arches, or securing the one or more dental arches to each other using elastics.

A 12^{th} aspect of the disclosure may further extend any of the 1^{st} through 11^{th} aspects of the disclosure. In the 12^{th} aspect of the disclosure, the one or more actions comprise adjusting at least one of a position or an orientation of one or more teeth on the one or more dental arches.

A 13^{th} aspect of the disclosure may further extend any of the 1^{st} through 12^{th} aspects of the disclosure. In the 13^{th} aspect of the disclosure, the one or more actions and the target state that are determined minimize at least one of a number of stages of the orthodontic treatment plan or a duration of orthodontic treatment performed according to the orthodontic treatment plan.

An 14^{th} aspect of the disclosure may further extend any of the 1^{st} through 13^{th} aspects of the disclosure. In the 14^{th} aspect of the disclosure, the method further comprises: determining an occlusion type for the one or more dental arches based on processing of the clinical data, wherein the occlusion type comprises at least one of an open bite, a cross bite, or a deep bite; wherein the one or more actions are determined based at least in part on the occlusion type.

A 15^{th} aspect of the disclosure may further extend any of the 1^{st} through 14^{th} aspects of the disclosure. In the 15^{th} aspect of the disclosure, the method further comprises: determining a type of malocclusion for the one or more dental arches based on processing of the clinical data; wherein the one or more actions are determined based at least in part on the type of malocclusion.

A 16^{th} aspect of the disclosure may further extend any of the 1^{st} through 15^{th} aspects of the disclosure. In the 16^{th} aspect of the disclosure, the one or more dental arches comprise an upper dental arch of the patient, the method further comprising: receiving, by the processing device, clinical data of a first state of a lower dental arch of the patient; and determining, by the processing device, one or more stages of an orthodontic treatment plan for correcting the lower dental arch based on processing of the clinical data, wherein determining a stage of the orthodontic treatment plan for correcting the lower dental arch comprises: determining one or more additional actions to be performed with respect to the lower dental arch; and determining a target state of the lower dental arch that is predicted to result at least in part from the one or more additional actions.

A 17^{th} aspect of the disclosure may further extend any of the 1^{st} through 16^{th} aspects of the disclosure. In the 17^{th} aspect of the disclosure, the method further comprises: determining a current relation between an upper dental arch of the one or more dental arches and a lower dental arch of the one or more dental arches, wherein determining the target state of the one or more dental arches that is predicted to result at least in part from the one or more actions comprises determining a target relation between the upper dental arch and the lower dental arch.

An 18^{th} aspect of the disclosure may further extend any of the 1^{st} through 17^{th} aspects of the disclosure. In the 18^{th} aspect of the disclosure, determining the one or more actions to be performed with respect to the one or more dental arches comprises determining one or more restorative dental actions to be performed, the one or more restorative dental actions associated with one or more restorative options.

A 19^{th} aspect of the disclosure may further extend the 18^{th} aspect of the disclosure. In the 19^{th} aspect of the disclosure, the one or more restorative options comprise at least one of a) one or more allowable dimensions for use of a veneer, b) a type of veneer to use, or c) a veneer thickness; and the one or more restorative dental actions to be performed comprise an amount of tooth mass to remove from a tooth that is to receive the veneer

20^{th} aspect of the disclosure may further extend the 18^{th} aspect of the disclosure. In the 20^{th} aspect of the disclosure, a space to place a dental implant is impinged upon by roots of one or more teeth that are adjacent to the space, and wherein the one or more restorative dental actions to be performed comprise spreading the roots of the one or more teeth apart to allow for placement of the dental implant in the space.

In a 21^{st} aspect of the disclosure, a method comprises: receiving, by a processing device, a training data item comprising clinical data of a state of one or more dental arches (and optionally a spatial relationship or bite relation between upper and lower dental arches) and information about performance of orthodontic treatment of the one or more dental arches; determining, by the processing device, one or more stages of an orthodontic treatment plan for correcting the one or more dental arches based on processing of the clinical data using a machine learning model, wherein determining a stage of the orthodontic treatment plan comprises: determining one or more actions to be performed with respect to the one or more dental arches; and determining a target state of the one or more dental arches that results at least in part from the one or more actions; determining a cost value or a reward value associated with the one or more stages of the orthodontic treatment plan using a cost function; and updating one or more nodes of the machine learning model based on the cost value or the reward value.

A 22^{nd} aspect of the disclosure may further extend the 21^{st} aspect of the disclosure. In the 22^{nd} aspect of the disclosure, the determining the cost value or the reward value and the updating the one or more nodes of the machine learning model are performed according to a reinforcement learning algorithm.

A 23^{rd} aspect of the disclosure may further extend the 27^{th} aspect of the disclosure. In the 23^{rd} aspect of the disclosure, the reinforcement learning algorithm is one of Q learning, deep Q learning, or double deep Q networks.

An 24^{th} aspect of the disclosure may further extend any of the 21^{st} through 23^{rd} aspects of the disclosure. In the 24^{th} aspect of the disclosure, the method further comprises: determining a number of stages of the orthodontic treatment plan; determining a delta between the number of stages of the orthodontic treatment plan and a target maximum number of stages for the orthodontic treatment plan; and determining the cost value or the reward value based on the delta.

A 25^{th} aspect of the disclosure may further extend any of the 21^{st} through 24^{th} aspects of the disclosure. In the 25^{th} aspect of the disclosure, the method further comprises: determining a predicted amount of time for completion of the orthodontic treatment plan; determining a delta between the predicted amount of time and a target maximum amount of time for completion of the orthodontic treatment plan; and determining the cost value or the reward value based on the delta.

A 26^{th} aspect of the disclosure may further extend any of the 21^{st} through 25^{th} aspects of the disclosure. In the 26^{th} aspect of the disclosure, the method further comprises: receiving an input of at least one of a target number of stages or a target amount of time for completion of the orthodontic treatment plan; and setting the cost function based on the input.

A 27^{th} aspect of the disclosure may further the 26^{th} aspect of the disclosure. In the 27^{th} aspect of the disclosure, the machine learning model is trained to generate an orthodontic treatment plan that achieves a target state of a dental arch while minimizing at least one of a number of stages or an amount of time for completion of the orthodontic treatment plan.

A 28^{th} aspect of the disclosure may further extend any of the 21^{st} through 27^{th} aspects of the disclosure. In the 28^{th} aspect of the disclosure, the clinical data is labeled with an occlusion type, wherein the occlusion type is one of an open bite, a deep bite, or a cross bite.

A 29^{th} aspect of the disclosure may further extend any of the 21^{st} through 28^{th} aspects of the disclosure. In the 29^{th} aspect of the disclosure, the clinical data is labeled with a treatment issue, wherein the treatment issue comprises at least one of a vertical issue, a sagittal issue, or a transverse issue.

A 30^{th} aspect of the disclosure may further extend any of the 21^{st} through 29^{th} aspects of the disclosure. In the 30^{th} aspect of the disclosure, the clinical data is labeled with a number of treatment stages, actions performed at one or more of the treatment stages, and a degree of success of the orthodontic treatment.

A 31^{st} aspect of the disclosure may further extend any of the 21^{st} through 30^{th} aspects of the disclosure. In the 31^{st} aspect of the disclosure, the machine learning model is trained to receive new clinical data of a patient's dental arch and to output a plurality of treatment plan options for orthodontic treatment of the patient's dental arch.

A 32^{nd} aspect of the disclosure may further extend any of the 21^{st} through 31^{st} aspects of the disclosure. In the 32^{nd} aspect of the disclosure, the training data item comprises multiple states of the one or more dental arches, each state of the multiple states associated with a different stage of orthodontic treatment.

A 33^{rd} aspect of the disclosure may further extend any of the 21^{st} through 32^{nd} aspects of the disclosure. In the 33^{rd} aspect of the disclosure, the clinical data comprises at least one of: one or more color two-dimensional (2D) images of the one or more dental arches, one or more three-dimensional (3D) models of the one or more dental arches, one or more intraoral scans of the one or more dental arches, or one or more x-ray images (e.g., radiographs) of the one or more dental arches.

A 34^{th} aspect of the disclosure may further extend any of the 21^{st} through 33^{rd} aspects of the disclosure. In the 34^{th} aspect of the disclosure, the one or more actions comprise at least one of widening a dental arch of the one or more dental arches, adding one or more attachments to the dental arch, extracting one or more teeth from the dental arch, performing interproximal reduction for one or more teeth of the dental arch, or securing the dental arch to an opposing dental arch using elastics.

A 35^{th} aspect of the disclosure may further extend any of the 21^{st} through 34^{th} aspects of the disclosure. In the 35^{th} aspect of the disclosure, the training data item further comprises clinical data of bite relation between an upper dental arch and a lower dental arch of the one or more dental arches.

A 36^{th} aspect of the disclosure may further extend any of the 1^{st} through or 35^{th} aspects of the disclosure. In the 36^{th} aspect of the disclosure, a computer readable medium comprises instructions that, when executed by a processing device, cause the processing device to perform the method of any of the 1^{st} through 35^{th} aspects of the disclosure.

A 37^{th} aspect of the disclosure may further extend any of the 1^{st} through or 35^{th} aspects of the disclosure. In the 37^{th} aspect of the disclosure, a system comprises: a computing device comprising a memory; and a processing device, wherein the processing device is to execute instructions from the memory to perform the method of any of the 1^{st} through or 35^{th} aspects of the disclosure.

A 38^{th} aspect of the disclosure may further extend the 37^{th} aspect of the disclosure. In the 38^{th} aspect of the disclosure, the computing device is configured to cause one or more orthodontic aligners to be manufactured in accordance with the one or more stages of the orthodontic treatment plan.

A 39^{th} aspect of the disclosure may further extend the 37^{th} or 38^{th} aspects of the disclosure. In the 39^{th} aspect of the disclosure, the system further comprises: an additional computing device configured to send the clinical data to the computing device; and a storage device configured to store the orthodontic treatment plan.

### BRIEF DESCRIPTION OF THE DRAWINGS

Embodiments of the present invention are illustrated by way of example, and not by way of limitation, in the figures of the accompanying drawings.

FIG. 1 illustrates one embodiment of a system for orthodontic treatment planning and implementation of orthodontic treatment, in accordance with an embodiment of the present disclosure.

FIG. 2 illustrates a model training workflow and a model application workflow for a treatment planning application, in accordance with an embodiment of the present disclosure.

FIG. 3A illustrates application of reinforcement learning to train one or more machine learning models to generate orthodontic treatment plans, in accordance with an embodiment of the present disclosure.

FIG. 3B illustrates an example machine learning model architecture, in accordance with an embodiment of the present disclosure.

FIG. 3C illustrates an example auto-encoder, in accordance with an embodiment of the present disclosure.

FIG. 4 illustrates a flow diagram for a method of performing reinforcement learning to train a machine learning model to perform treatment planning, in accordance with an embodiment.

FIG. 5 illustrates a flow diagram for a method of training a machine learning model to generate an orthodontic treatment plan, in accordance with an embodiment.

FIG. 6 illustrates a flow diagram for a method of determining a cost value or reward value to apply for one or more stages of treatment of a generated treatment plan.

FIG. 7 illustrates a flow diagram for a method of generating an orthodontic treatment plan using a trained machine learning model, in accordance with an embodiment.

FIG. 8 illustrates a flow diagram for a method of selecting a trained machine learning model to generation of an orthodontic treatment plan, in accordance with an embodiment.

FIG. 9 illustrates a flow diagram for a method of determining actions for one or more stages of an orthodontic treatment plan, in accordance with an embodiment.

FIG. 10A illustrates a tooth repositioning system, according to certain embodiments.

FIG. 10B illustrates a method of orthodontic treatment using a plurality of appliances, according to certain embodiments.

FIG. 11 illustrates a method for designing an orthodontic appliance, according to certain embodiments.

FIG. 12 illustrates a method for digitally planning an orthodontic treatment, according to certain embodiments.

FIG. 13 illustrates a block diagram of an example computing device, in accordance with embodiments of the present invention.

### DETAILED DESCRIPTION

Described herein are methods and apparatuses for determining one or more aspects of an orthodontic treatment plan using machine learning. In embodiments, clinical data representing a current state of one or more patient dental arches and/or one or more past states of the one or more patient dental arches (e.g., including a spatial relationship or bite relation between the patient's upper and lower dental arches) are input into a trained machine learning model. The trained machine learning model may then output a full treatment plan (e.g., a full orthodontic treatment plan), one or more stages of a treatment plan, recommendations of a treatment plan, etc. In embodiments, the machine learning model is trained to learn a policy for achieving clinical (e.g., orthodontic and/or restorative) targets while maximizing one or more defined rewards or goals (e.g., minimizing a number of aligners). The machine learning model may be trained, for example, to perform clinical judgment as to generation of a new treatment plan (e.g., orthodontic treatment plan, restorative treatment plan or ortho-restorative treatment plan) or updating an existing treatment plan. In some embodiments, a restorative treatment plan or orthodontic treatment plan may be updated to an ortho-restorative treatment plan. For one or more of the stages of an orthodontic treatment plan or ortho-restorative treatment plan, the trained machine learning model may output one or more actions to be performed, such as interproximal reduction, tooth extraction, addition of attachments to one or more teeth, use of elastics, widening of one or more dental arch (e.g., via a palatal expander), increasing space between two or more teeth, repositioning one or more teeth on one or more directions, rotating one or more teeth about one or more axes, and so on. In embodiments the trained machine learning model may suggest actions that do not require doctor intervention and/or may suggest clinical actions for a doctor to perform, and may supplement a doctor's decision making with such suggested actions. In embodiments, the machine learning model may be trained using reinforcement learning based on a library of prior successful and/or unsuccessful treatments of past patients. The machine learning model may have been trained to optimize for one or more goals, such as a shortest treatment time while still achieving a target end state of a patient's dentition, a minimal number of treatment stages while still achieving the target end state of the patient's dentition, and so on. In embodiments, the machine learning model may be trained to satisfy both clinical constraints (e.g., avoiding collisions of teeth and/or roots, tooth movement constraints, etc.) and business constraints (e.g., number of treatment stages, length of treatment, etc.).

Orthodontic treatment is typically performed in a sequence of stages, and an orthodontic treatment plan may call for a patient's teeth to move by a specified amount for each of the stages. However, it can be difficult for a dental practitioner to determine a number of stages to use to achieve a target goal, to determine what action or actions to perform on the patient's teeth, when to perform those actions, whether treatment is progressing according to plan, and/or what actions to perform once a determination is made that treatment is not progressing according to plan. For example, it can be difficult for the dental practitioner to determine whether the teeth are moving according to the treatment plan, whether some of the teeth are not moving according to the treatment plan, whether a planned class of occlusion will be achieved, whether a planned arch expansion is achieved, whether to extract any teeth, whether to perform interproximal reduction (IPR), whether to use elastics between the upper and lower dental arch, where to place attachments on teeth, and so on. Some embodiments are discussed with respect to orthodontic treatment. However, it should be understood that such embodiments also apply to ortho-restorative treatment, where ortho-restorative treatment includes both orthodontic treatment and restorative treatment.

Embodiments provide a method and system for using machine learning to assist treatment planning, and in particular to suggest actions associated with a treatment plan and/or for a doctor to take at one or more stages of orthodontic treatment to achieve one or more goals, such as a minimal number of treatment stages, a shortest possible treatment time, a minimal amount of patient discomfort, and so on. In embodiments the system and method are additionally capable of assessing the actual progress of an orthodontic treatment plan that has a target end position (e.g., of assessing a patient's teeth during intermediate stages of a multi-stage orthodontic treatment plan), and updating the treatment plan and/or suggesting one or more actions for the treatment plan based on such assessment. Suggested actions may include corrective actions such as modifications to the final treatment plan (e.g., to final teeth positions) and/or staging of the teeth positions in the treatment plan (if the treatment plan is a multi-stage treatment plan). Staging refers to the sequence of movements from current or initial teeth positions to new teeth positions. Staging includes determining which tooth movements will be performed at different phases of treatment. Some corrective actions may require one or more actions or operations to be performed by the dental practitioner, such as placement of attachments, IPR, application of a palatal expander, use of a temporary anchorage device, and so on.

Embodiments provide significant advantages over traditional treatment techniques in orthodontics, and can improve treatment results of orthodontic treatment while shortening treatment time, reducing a number of stages used, and so on. Embodiments may provide a system that suggests stages of orthodontic treatment, notifies a dental practitioner of suggested actions to perform at one or more stages of treatment, and so on. Accordingly, treatment plan efficacy is improved in embodiments. Such improvements in treatment plan efficacy are likely to result in increased patient satisfaction as well as reduced costs by reducing the number of consecutive refinements that are made to a treatment plan (and associated orders of additional aligners) during treatment.

Embodiments are discussed herein with reference to multi-stage treatment plans. However, such embodiments also apply to single stage orthodontic treatment plans that have a target end position. For example, image data may be generated some time after beginning a single stage orthodontic treatment plan. If the image data shows that progress of the single stage treatment plan is not as expected, then the target end position may be adjusted for the single stage treatment plan and/or one or more treatment parameters for reaching the target end position may be adjusted. Accordingly, it should be understood that all discussion of multi-stage treatment plans herein also applied to single stage treatment plans with target end positions and/or conditions.

Doctors have noted for years that control of upper lateral teeth is very difficult. Embodiments of the present disclosure, when applied to this prevalent clinical problem, may help to improve control of these upper lateral teeth.

Furthermore, some embodiments are discussed herein with reference to generation and use of aligners. As used herein, an aligner is an orthodontic appliance that is used to reposition teeth. It should be noted that embodiments also apply to other types of orthodontic appliances including but not limited to brackets and wires, retainers, or functional appliances. For example, determined actions at one or more stages of orthodontic treatment using brackets and wires may include bonding brackets to particular locations of teeth, selection of arch wires, selection of a type of ligatures, a change in arch wire selection, repositioning one or more brackets, bending arch wires at specified times, and so on. Accordingly, it should be understood that any discussion of aligners herein also applies to other types of orthodontic appliances.

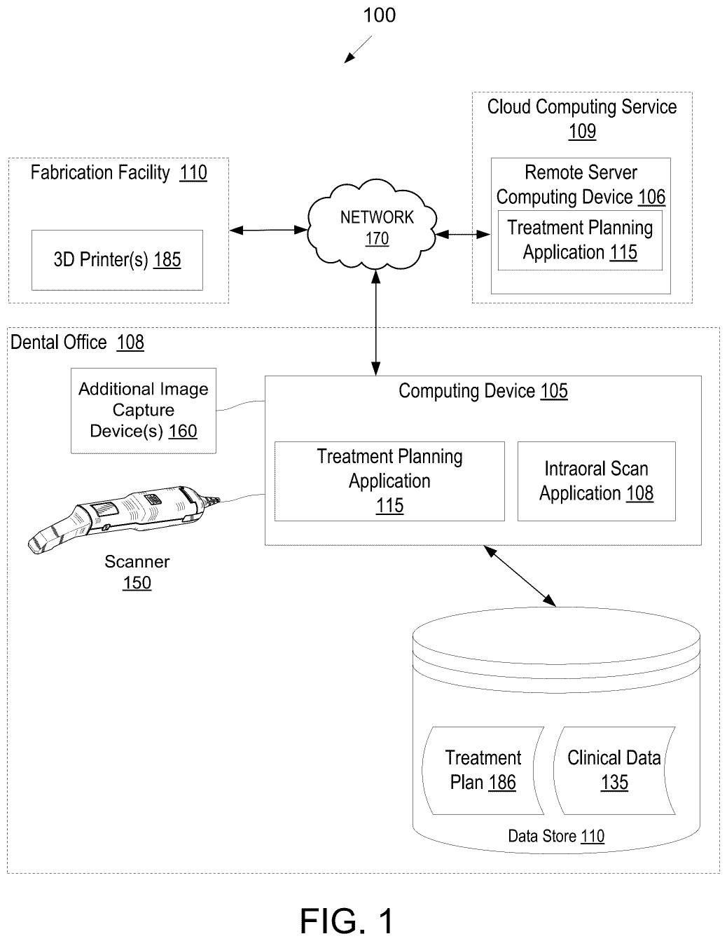

FIG. 1 illustrates one embodiment of a system 100 for performing orthodontic treatment or ortho-restorative treatment. In one embodiment, system 100 carries out one or more operations of below described in methods 200-1200. System 100 may include only components located at a single location (e.g., at a dentist office 108 or a dental lab) or may include components that are at multiple different locations (e.g., components at dental office 108 or dental lab and components at a second location that is remote from the dental office, such as a server farm that provides a cloud computing service 109). The system 100 may optionally also include a fabrication facility 110 including one or more three-dimensional (3D) printers 185 and/or other additive manufacturing machines that may be used to manufacture aligners. The dental office 108 includes a computing device 105. In embodiments that incorporate use of a cloud computing service 109 or other remote server, system 100 further includes a remote server computing device 109 connected to computing device 105 via a network 170. The network 170 may be a local area network (LAN), a public wide area network (WAN) (e.g., the Internet), a private WAN (e.g., an intranet), or a combination thereof.

Computing device 105 may be coupled to and/or include a data store 110. Computing device 106 may also be connected to and/or include a data store (not shown). The data stores may be local data stores and/or remote data stores. Computing device 105 and computing device 106 may each include one or more processing devices, memory, secondary storage, one or more input devices (e.g., such as a keyboard, mouse, tablet, and so on), one or more output devices (e.g., a display, a printer, etc.), and/or other hardware components. In some embodiments, computing device 105 and/or computing device 106 does not include input and/or output devices (e.g., is not connected to a keyboard, a mouse, a display, etc.). The computing device 105 may be integrated into the scanner 150 or image capture device 160 in some embodiments to improve mobility.

In some embodiments, a scanner 150 for obtaining three-dimensional (3D) data of a dental site in a patient's oral cavity is operatively connected to the computing device 105. In embodiments, one or more handheld intraoral scanner 150 (also referred to as an intraoral scanner or simply a scanner) is wirelessly connected to computing device 105. In one embodiment, scanner 150 is wirelessly connected to computing device 105 via a direct wireless connection. In one embodiment, scanner 150 is wirelessly connected to computing device 105 via a wireless network. Alternatively, scanner 150 may be connected to a computing device 105 via a wired connection.

Scanner 150 may include a probe (e.g., a hand held probe) for optically capturing three dimensional structures. One example of such a scanner 150 is the iTero® intraoral digital scanner manufactured by Align Technology, Inc. In some embodiments, scanner 150 corresponds to an intraoral scanner as described in U.S. Publication No. 2019/0388193, filed Jun. 19, 2019, entitled “Intraoral 3D Scanner Employing Multiple Miniature Cameras and Multiple Miniature Pattern Projectors,” which is incorporated by reference herein. In some embodiments, scanner 150 corresponds to an intraoral scanner as described in U.S. application Ser. No. 16/910,042, filed Jun. 23, 2020 and entitled “Intraoral 3D Scanner Employing Multiple Miniature Cameras and Multiple Miniature Pattern Projectors,” which is incorporated by reference herein. In some embodiments, scanner 150 corresponds to an intraoral scanner as described in U.S. Pat. No. 10,835,128, issued Nov. 17, 2020, which is incorporated by reference herein. In some embodiments, scanner 150 corresponds to an intraoral scanner as described in U.S. Pat. No. 10,918,286, issued Feb. 21, 2021, which is incorporated by reference herein.

Intraoral scanner 150 may generate intraoral scans, which may be or include color or monochrome 3D information, and send the intraoral scans to computing device 105. In some embodiments, intraoral scans include height maps. Intraoral scanner 150 may additionally or alternatively generate color two-dimensional (2D) images (e.g., viewfinder images), and send the color 2D images to local server computing device 105. Scanner 150 may additionally or alternatively generate 2D or 3D images under certain lighting conditions, such as under conditions of infrared or near-infrared (NIRI) light and/or ultraviolet light, and may send such 2D or 3D images to server computing device 105. Intraoral scans, color images, and images under specified lighting conditions (e.g., NIRI images, infrared images, ultraviolet images, etc.) are collectively referred to as intraoral scan data. Intraoral scan data and other data of dental arches, such as x-ray images (also referred to herein as radiographs), 2D or 3D images generated by a device other than an intraoral scanner, cone beam computed tomography (CBCT) scan data, etc. are collectively referred to as clinical data 135. Clinical data may include images, etc. of only a single dental arch or of both the upper and lower dental arch of a patient. For clinical data that includes information for both the upper and lower dental arches, the clinical data may include bite relation data (also referred to as spatial relationships) between the upper and lower dental arches. Such information may indicate how the upper and lower dental arches articulate relative to one another and/or occlusal contact information between teeth in the upper and lower dental arches. An operator may start recording scans with the scanner 150 at a first position in the oral cavity, move the scanner 150 within the oral cavity to a second position while the scans are being taken, and then stop recording the scans. In some embodiments, recording may start automatically as the scanner 150 identifies teeth and/or other objects.

An intraoral scan application 108 running on computing device 105 may communicate with the scanner 150 to effectuate an intraoral scan. A result of the intraoral scan may be clinical data 135 that may include one or more sets of intraoral scans, one or more sets of viewfinder images (e.g., color 2D images showing a field of view of the intraoral scanner), one or more sets of NIRI images, and so on. Each intraoral scan may be a two-dimensional (2D) or 3D image that includes a height information (e.g., a height map) of a portion of a dental site, and thus may include x, y and z information. In one embodiment, each intraoral scan is a point cloud. In one embodiment, the intraoral scanner 150 generates numerous discrete (i.e., individual) intraoral scans and/or additional images. In some embodiments, sets of discrete intraoral scans may be merged into a smaller set of blended intraoral scans, where each blended scan is a combination of multiple discrete intraoral scans.

In embodiments, scanner 150 generates and sends to computing device 105 a stream of intraoral scan data. The stream of intraoral scan data may include separate streams of intraoral scans, color images and/or NIRI images (and/or other images under specific lighting conditions) in some embodiments. In one embodiment, a stream of blended intraoral scans is sent to computing device 105.

Computing device 105 receives intraoral scan data from scanner 150, then stores the intraoral scan data in data store 110 as clinical data 135. According to an example, a user (e.g., a practitioner) may subject a patient to intraoral scanning. In doing so, the user may apply scanner 150 to one or more patient intraoral locations. The scanning may be divided into one or more segments. As an example, the segments may include a lower buccal region of the patient, a lower lingual region of the patient, a upper buccal region of the patient, an upper lingual region of the patient, one or more preparation teeth of the patient (e.g., teeth of the patient to which a dental device such as a crown or an orthodontic alignment device will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with scan being directed towards an interface area of the patient's upper and lower teeth). Via such scanner application, the scanner 150 may provide intraoral scan data to computing device 105. The intraoral scan data may be provided in the form of intraoral scan/image data sets, each of which may include 2D intraoral scans/images and/or 3D intraoral scans/images of particular teeth and/or regions of an intraoral site. In one embodiment, separate scan/image data sets are created for the maxillary arch, for the mandibular arch, for a patient bite, and for each preparation tooth. Alternatively, a single large intraoral scan/image data set is generated (e.g., for a mandibular and/or maxillary arch). Such scans/images may be provided from the scanner to the computing device 105 in the form of one or more points (e.g., one or more pixels and/or groups of pixels). For instance, the scanner 150 may provide such a 3D scan/image as one or more point clouds.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Additionally, the manner in which the oral cavity is to be scanned may depend on a doctor's scanning preferences and/or patient conditions.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

During an intraoral scan session, intraoral scan application 108 receives and processes intraoral scan data (e.g., intraoral scans) and generates a 3D surface of a scanned region of an oral cavity (e.g., of a dental site) based on such processing. To generate the 3D surface, intraoral scan application 108 may register and “stitch” or merge together the intraoral scans generated from the intraoral scan session in real time or near-real time as the scanning is performed. In one embodiment, performing registration includes capturing 3D data of various points of a surface in multiple scans (views from a camera), and registering the scans by computing transformations between the scans. The 3D data may be projected into a 3D space for the transformations and stitching. The scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). In one embodiment, registration is performed using blended scans and/or reduced or cropped scans. Registration algorithms are carried out to register two or more adjacent intraoral scans and/or to register an intraoral scan with an already generated 3D surface, which essentially involves determination of the transformations which align one scan with the other scan and/or with the 3D surface. Registration may involve identifying multiple points in each scan (e.g., point clouds) of an scan pair (or of a scan and the 3D model), surface fitting to the points, and using local searches around points to match points of the two scan (or of the scan and the 3D surface). For example, intraoral scan application 115 may match points of one scan with the closest points interpolated on the surface of another image, and iteratively minimize the distance between matched points. Other registration techniques may also be used. Intraoral scan application 115 may repeat registration and stitching for all scans of a sequence of intraoral scans and update the 3D surface as the scans are received.

When a scan session is complete (e.g., all scans for an intraoral site or dental site have been captured), intraoral scan application 108 or treatment planning application 115 may generate a virtual 3D model (also referred to as a digital 3D model) of one or more scanned dental sites. The virtual 3D model includes a 3D surface of the one more scanned dental sites, but has a higher degree of accuracy than the 3D surface generated during the scanning process. To generate the virtual 3D model, intraoral scan application 108 or treatment planning application 115 may register and “stitch” or merge together the intraoral scans generated from the intraoral scan session. In one embodiment, registration is performed for adjacent and/or overlapping intraoral scans (e.g., each successive frame of an intraoral video). In one embodiment, registration is performed using blended scans and/or reduced or cropped scans. Registration algorithms may be carried out to register two or more adjacent intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 108 or treatment planning application 115 may match points of one scan with the closest points interpolated on the surface of another scan, and iteratively minimize the distance between matched points. Other registration techniques may also be used. The registration and stitching that are performed to generate the 3D model may be more accurate than the registration and stitching that are performed to generate the 3D surface that is shown in real time or near-real time during the scanning process.

Intraoral scan application 108 or treatment planning application 115 may repeat registration for all scans of a sequence of intraoral scans to obtain transformations for each scan, to register each scan with the previous one and/or with a common reference frame (e.g., with the 3D model). Intraoral scan application 108 or treatment planning application 115 integrates all scans into a single virtual 3D model by applying the appropriate determined transformations to each of the scans. Each transformation may include rotations about one to three axes and translations within one to three planes.

In addition to clinical data 135 including data captured by scanner 150 and/or data generated from such captured data (e.g., a virtual 3D model), clinical data 135 may also or alternatively include data from one or more additional image capture devices 160. The additional image capture devices 160 may include an x-ray device capable of generating standard x-rays (e.g., bite wing x-rays), panoramic x-rays, cephalometric x-rays, and so on. The additional image capture devices 160 may additionally or alternatively include an x-ray device capable of generating a cone beam computed tomography (CBCT) scan. Additionally, or alternatively, the additional image capture devices 160 may include a standard optical image capture device (e.g., a camera) that generates two-dimensional or three-dimensional images or videos of a patient's oral cavity and dental arch. For example, the additional image capture device 160 may be a mobile phone, a laptop computer, an image capture accessory attached to a laptop or desktop computer (e.g., a device that uses Intel® RealSense™ 3D image capture technology), and so on. Such an additional image capture device 160 may be operated by a patient or a friend or family of the patient, and may generate 2D or 3D images that are sent to the computing device 105 or computing device 109 via network 170. Accordingly, clinical data 135 may include 2D optical images, 3D optical images, virtual 2D models, virtual 3D models, intraoral scans, 2D x-ray images, 3D x-ray images, and so on.

Once intraoral scanning is complete, treatment planning application 115 may receive intraoral scan data and/or a 3D model generated based on the intraoral scan data. If a 3D model of the upper and lower dental arches have not been generated, treatment planning application 115 may generate the 3D model(s) as described above. Treatment planning application 115 may then input clinical data 135, which may include the 3D model(s), intraoral scans, 2D images, 3D images, projections of the 3D model(s) onto one or more planes, CBCT scans, x-ray images, and/or other data into a trained machine learning model that has been trained to facilitate generation of an orthodontic treatment plan. In some embodiments, a final target state of a patient's dentition (e.g., a 3D model of the patient's dental arch(es) post treatment) is generated, and the clinical data 135 input into the trained machine learning model further includes the information on the final target state of the patient's dentition. For example, a doctor may determine the final target dentition, and a 3D model of the patient's upper and/or lower dental arches may be generated and input into the trained machine learning model along with the current state of the patient's dentition. In embodiments, the trained machine learning model outputs a complete orthodontic treatment plan or one or more stages of the orthodontic treatment plan 186. The machine learning model may additionally output one or more actions to be performed at one or more stages of treatment, such as tooth extraction, IPR, palatal expansion, addition of attachments, use of elastics, moving one or more teeth in a sagittal, transverse and/or vertical plane, rotating one or more teeth about one or more axis, changing a thickness and/or shape of an aligner, adding strengthening features (e.g., dimples) to an aligner, and so on. The treatment plan 186 may be stored in data store 110 in embodiments.

In embodiments, the intraoral scan is performed during an intermediate stage of a multi-stage orthodontic treatment plan 186. Alternatively, or additionally, other clinical data 135 may be generated at a start of a multi-stage orthodontic treatment plan (e.g., before the treatment plan has been generated). In embodiments, one or more 2D images of a patient's dentition are received during an intermediate stage of treatment. These images may have been generated, for example, by a user device, such as a mobile phone of the patient or someone else associated with the patient.

The multi-stage orthodontic treatment plan 186 may be for a multi-stage orthodontic treatment or procedure (or a multi-stage ortho-restorative treatment). The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, aligners, or functional appliances. Different aligners may be formed for each treatment stage to provide forces to move the patient's teeth. The shape of each aligner is unique and customized for a particular patient and a particular treatment stage. The aligners each have teeth-receiving cavities that receive and resiliently reposition the teeth in accordance with a particular treatment stage.

Restorative dental treatment refers to dental procedures and practices aimed at restoring the function, integrity, and appearance of damaged, decayed, or missing teeth. The primary goal of restorative dentistry is to repair or replace teeth to improve oral health and functionality, allowing patients to chew, speak, and smile with confidence. Common restorative dental procedures include applying fillings, applying crowns or caps, applying bridges, applying dentures, applying dental implants (e.g., metal posts or frames surgically placed into the jawbone that serve as a base for replacement teeth), applying inlays and onlays, applying veneers, and root canal therapy.

In some cases, a machine learning model is used to generate some or all of a new orthodontic treatment plan or ortho-restorative treatment plan. In some cases, a multi-stage orthodontic treatment plan 186 for a patient may have initially been generated by a dental practitioner (e.g., an orthodontist) or by treatment planning application 115 after performing a scan of an initial pre-treatment condition of the patient's dental arch and updates to the treatment plan may be determined. The treatment plan 186 may also begin at home (based on a patient scan of himself) or at a scanning center. The treatment plan 186 might be created automatically (e.g., by treatment planning application 115) or by a professional (including an Orthodontist) in a remote service center. Intraoral scans may provide surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.). Clinical data from the initial intraoral scan may be used to generate a virtual three-dimensional (3D) model or other digital representation of the initial or starting condition for the patient's upper and/or lower dental arches.

The dental practitioner or treatment planning application 115 may then determine a desired final condition for the patient's dental arch. The final condition of the patient's dental arch may include a final arrangement, position, orientation, etc. of the patient's teeth, and may additionally include a final bite position, a final occlusion surface, a final arch length, and so on. In embodiments, the final condition is of an upper and lower dental arch of the patient, and includes a final bite relation of the upper and lower dental arches. A movement path of some or all of the patient's teeth and the patient bite changes from starting positions to planned final positions may then be calculated or determined by treatment planning application 115. In some embodiments, the movement path is calculated using one or more suitable computer programs, which can take digital representations of the initial and final positions as input, and provide a digital representation of the movement path as output. In some embodiments, the movement path for one or more stages of treatment is determined by a trained machine learning model. The movement path for any given tooth may be calculated based on the positions and/or movement paths of other teeth in the patient's dentition. For example, the movement path can be optimized based on minimizing the total distance moved, preventing collisions between teeth, avoiding tooth movements that are more difficult to achieve, or any other suitable criteria. In some instances, the movement path can be provided as a series of incremental tooth movements that, when performed in sequence, result in repositioning of patient's teeth from the starting positions to the final positions, and/or that result in a final bite relation between the patient's upper and lower dental arches.

Multiple treatment stages may be generated based on the determined movement path by treatment planning application 115 using application of machine learning. Each of the treatment stages can be incremental repositioning stages of an orthodontic treatment procedure designed to move one or more of the patient's teeth from a starting tooth arrangement for that treatment stage to a target arrangement for that treatment stage for one or both dental arches. A different 3D model of a target condition for a treatment stage may be generated by the treatment planning application 115 for each of the treatment stages, and for each dental arch.

One or a set of orthodontic appliances (e.g., aligners) are then fabricated at fabrication facility 110 based on the generated treatment stages (e.g., based on the 3D models of the target conditions for each of the treatment stages). For example, a set of appliances can be fabricated, each shaped to accommodate a tooth arrangement specified by one of the treatment stages, such that the appliances can be sequentially worn by the patient to incrementally reposition the teeth from the initial arrangement to the target arrangement. The configuration of the aligners can be selected to elicit the tooth movements specified by the corresponding treatment stage. In some embodiments, 3D printers 185 print molds for dental arches at multiple stages of treatment based on 3D models, and polymeric sheets are thermoformed over the molds to form aligners. In some embodiments, 3D printers 185 directly print aligners based on 3D models of aligners determined based on 3D models of dental arches of a patient at multiple stages of treatment.

Sometimes it can be difficult to determine at the beginning of an orthodontic treatment what the final treatment plan should be. There are multiple reasons for this. For example, in some cases teeth do not move as expected due to the specific biology of a particular patient. Additionally, sometimes patient compliance with a treatment plan is sub-optimal (e.g., the patient does not wear his or her aligners as instructed). Accordingly, it can be beneficial in some embodiments to generate more than one treatment plan prior to beginning treatment and/or to generate multiple treatment plan update options during treatment. In some embodiments, a set of multi-stage orthodontic treatment plans is generated before treatment is initiated. The set of treatment plans may include a first treatment plan that has optimal target conditions. The first treatment plan may be a most aggressive treatment plan of the set (e.g., may call for the most movement of teeth out of any of the treatment plans in the set). The set of treatment plans may additionally include treatment plans that are less aggressive (e.g., that have alternative target conditions that include less change to the dental arch) and/or other treatment plans that are more aggressive. Additionally, or alternatively, the first treatment plan may call for fewer physical operations or procedures on the patient's mouth, and other treatment plans may call for more physical operations or procedures. For example, the first treatment plan may call for generation of a 5 mm arch length increase by distalization of the patient's molars, while a second treatment plan may call for generation of a 4 mm arch length increase by distalization of the patient's molars and additionally call for interproximal reduction or a tooth extraction.

The clinical data 135 received during the intermediate stage in the multi-stage orthodontic treatment plan 186 or ortho-restorative treatment plan as well as previous clinical data generated during one or more prior stages of the orthodontic treatment plan 186 and/or a stored state of the orthodontic treatment plan may be processed by treatment planning application 115 (e.g., by a trained machine learning model of treatment planning application 115). Based on the processing, treatment planning application 115 may determine one or more updated treatment stages and/or one or more actions associated with the one or more treatment stages. For example, treatment planning application 115 may determine one or more corrective actions, such as performance of IPR, extraction of a tooth, addition of attachments to one or more teeth, update to a planned position and/or orientation of one or more teeth, addition of further stages of treatment to a treatment plan, changing a shape of one or more aligners, and so on. Additionally, or alternatively, treatment planning application may add one or more restorative dental actions to one or more treatment stages. For example, a restorative workflow may be added at a certain stage of orthodontic treatment or between two stages of orthodontic treatment.

In some embodiments, computing device 105 offloads one or more operations to remote server computing device 106. Remote server computing device 106 may be provided by a cloud computing service 109, such as Amazon Web Services (AWS). Remote server computing device 106 may have increased resources such as memory resources, processing resources, and so on as compared to local server computing device 105. Remote server computing device 106 may include a version of treatment planning application 115, which may perform some or all of the operations previously described. In an embodiment, computing device 105 may send clinical data 135 to remote computing device 106, which may execute treatment planning application 115. Once a treatment plan has been generated, remote computing device may send instructions to fabrication facility 110 to cause one or more orthodontic aligners associated with stages of the orthodontic treatment plan to be manufactured. Additionally, or alternatively, the remote server computing device 106 may store the treatment plan in data store 110 and/or in another data store.

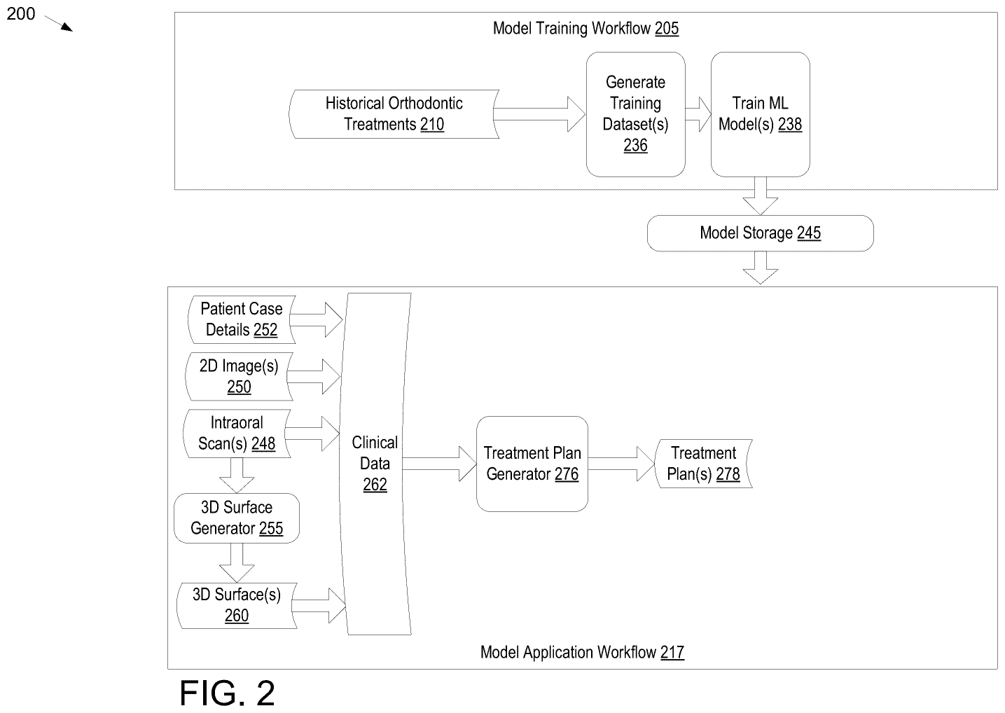

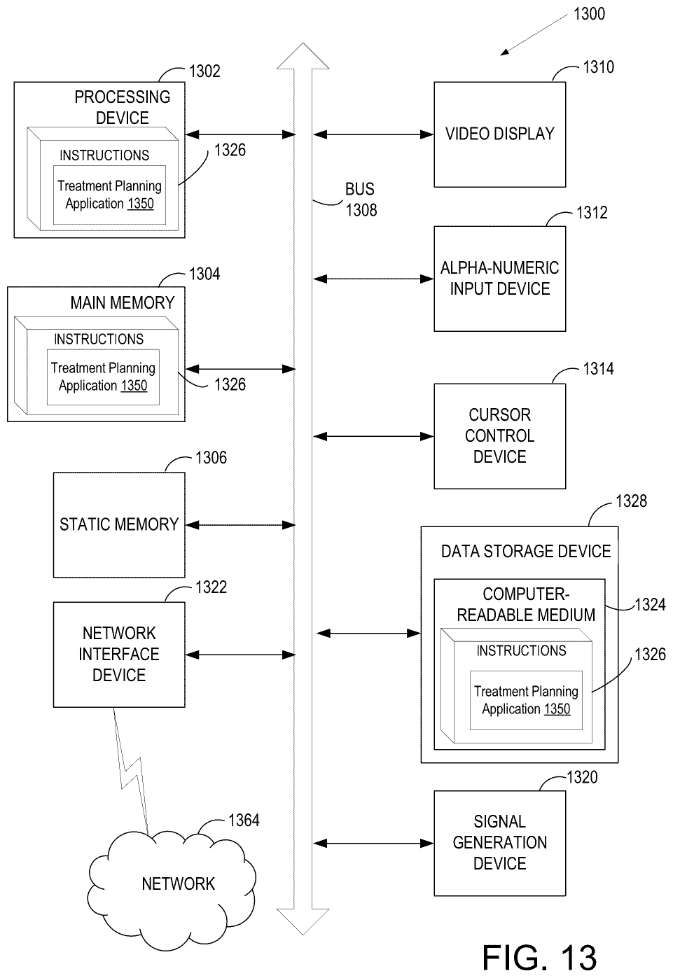

FIG. 2 illustrates a model training workflow 205 and a model application workflow 217 for a treatment planning application, in accordance with an embodiment of the present disclosure. In embodiments, the model training workflow 205 may be performed at a server which may or may not include a treatment planning application, and the trained models are provided to a treatment planning application (e.g., on computing device 105 or 106 of FIG. 1), which may perform the model application workflow 217. The model training workflow 205 and the model application workflow 217 may be performed by processing logic executed by a processor of a computing device. One or more of these workflows 205, 217 may be implemented, for example, by one or more machine learning modules implemented in a treatment planning application 115 or other software and/or firmware executing on a processing device of computing device 1300 shown in FIG. 13.

The model training workflow 205 is to train one or more machine learning models (e.g., deep learning models) to perform one or more prediction, recommendation or generation tasks with regards to an orthodontic treatment plan or ortho-restorative treatment plan based on clinical data (e.g., 3D scans, height maps, 2D color images, NIRI images, 3D surfaces generated based on intraoral scan data, 3D models of dental arches, etc.). The model application workflow 217 is to apply the one or more trained machine learning models to perform the prediction, recommendation or generation tasks with regards to an orthodontic treatment plan based on clinical data. One or more of the machine learning models may receive and process 3D data (e.g., 3D point clouds, 3D surfaces, portions of 3D models, etc.). One or more of the machine learning models may receive and process 2D data (e.g., 2D images, height maps, projections of 3D surfaces onto planes, etc.), which may be for a single dental arch or for both an upper and lower dental arches.

One type of machine learning model that may be used to perform some or all of the above asks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

For the model training workflow 205, a training dataset containing hundreds, thousands, tens of thousands, hundreds of thousands or more sets of clinical data (e.g., including intraoral scans, images and/or 3D models, etc.) should be used to form a training dataset. In embodiments, up to millions of cases of patient dentition that may have underwent an orthodontic procedure may be available for forming a training dataset, where each case may include various labels of one or more types of useful information. Each case may include, for example, data showing a 3D model, intraoral scans, height maps, color images, NIRI images, etc. of one or more dental sites, data showing pixel-level segmentation of the data (e.g., 3D model, intraoral scans, height maps, color images, NIRI images, etc.) into various dental classes (e.g., tooth, restorative object, gingiva, moving tissue, upper palate, etc.), data showing one or more assigned classifications for the data, data indicating a number of stages of orthodontic treatment that were used, data indicating a length of time for orthodontic treatment, data indicating a degree of success of orthodontic treatment, and so on. This data may be processed to generate one or multiple training datasets 236 for training of one or more machine learning models. The machine learning models may be trained, for example, to generate one more recommendations with respect to a treatment plan, to generate a treatment plan, to generate one or more stages of a treatment plan, to suggest actions to be performed at one or more stages of a treatment plan, and so on. Such trained machine learning models can be added to a treatment planning application, and can be applied to facilitate generation of orthodontic treatment plans.

In one embodiment, generating one or more training datasets 236 includes gathering one or more clinical data of historical orthodontic treatments (e.g., with labels) 210 and/or one or more 3D models. Processing logic may gather a training dataset 236 comprising clinical data from historical orthodontic treatments 210. To effectuate training, processing logic inputs the training dataset(s) 236 into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the images, scans or 3D surfaces (or data from the images, scans or 3D surfaces) into the machine learning model one at a time. Each input may include clinical data, which may include data from an image, intraoral scan or 3D surface in a training data item from the training dataset. As discussed above, training data items may also include other types of data such as color images, images generated under specific lighting conditions (e.g., UV or IR radiation), and so on. In embodiments, training data items include 2D or 3D representations of a current or initial state of a patient's dental arch(es) and a target final state of the patient's dental arch(es).

The machine learning model processes the input to generate an output. An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. Accordingly, the output may include one or more prediction, action, recommendation, treatment stage, full treatment plan, etc.

Processing logic may then compare the generated output to one or more values (e.g., one or more labels included in the training data item). In some embodiments, reinforcement learning is used to train the machine learning model according to a learning policy for treating orthodontic cases, as described in greater detail below. Accordingly, the output may be processed according to a reward function or cost function to determine a cost or reward associated with the output. The determined cost or reward may be used to adjust weights of one or more nodes in the machine learning model. An error term or delta may be determined for each node in the artificial neural network based on the cost or reward value that was computed. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

Once one or more trained ML models 238 are generated, they may be stored in model storage 245, and may be added to a treatment planning application (e.g., treatment planning application 115). Treatment planning application 115 may then use the one or more trained ML models 238 as well as additional processing logic to automate one or more aspects of treatment planning generation and/or to assist doctors in designing treatment plans and/or updating treatment plans that are not proceeding according to plan.

In one embodiment, model application workflow 217 includes one or more trained machine learning models that function as a treatment plan generator 276. Alternatively, or additionally, the one or more trained machine learning models may function as a treatment action recommender, a partial treatment plan generator, a treatment plan updater and/or a treatment plan assessor. For example, treatment plan generator 276 may be a deep neural network trained to generate treatment plans and/or information associated with treatment plans (e.g., actions, stages, recommendations, etc. for treatment plans).

For model application workflow 217, according to one embodiment, an intraoral scanner generates a sequence of intraoral scans 248 of one or both dental arches. A 3D surface generator 255 may perform registration between these intraoral scans, stitch the intraoral scans together, and generate a 3D surface or model 260 from the intraoral scans for the upper and/or lower dental arches. The intraoral scan(s) 248, 3D surface(s) 260 and/or other information such as 2D images 250 (e.g., as generated by an intraoral scanner or another device), 3D images, x-ray images, CBCT scans, panoramic x-ray images, patient case details 252 (e.g., indicating a level of malocclusion, indicating one or more missing teeth, indicating bite relation, indicating a type of treatment to be performed (e.g., aligners or braces/brackets), patient age, patient gender, etc.) may constitute clinical data 262.

Clinical data 262 may also include previously generated intraoral scans and/or 3D surfaces/models, a previously generated treatment plan, an indication of a current stage of treatment associated with a previously generated treatment plan, a saved state of a treatment plan (e.g., as previously output by treatment plan generator 276), a target final state of a patient's upper and/or lower dental arches (e.g., in the form of one or more 3D models), an indication of whether a patient is experiencing pain, and/or other information. Some or all of the clinical data 262 may be input into treatment plan generator 276, which may include a trained neural network. Based on the input clinical data, the machine learning model may identify, for example, one or more clinical issues (e.g., class or type of malocclusion), may identify an amount of tooth crowding in one or more areas, may identify whether clinical issues are present in a vertical, sagittal and/or transverse plane, may identify an improper or suboptimal bite relation, and so on. Based on the clinical data 262, treatment plan generator 276 outputs information associated with orthodontic treatment of a patient, restorative treatment of the patient and/or ortho-restorative treatment of the patient, such as one or more treatment plans 278.

In embodiments, a generated treatment plan may include a sequence of stages, and may include one or more actions (e.g., some of which are to be performed by a doctor) at one or more stages of treatment. Examples of actions include slowing down a velocity of tooth movement (e.g., reduce an amount of tooth movement between stages for a preexisting treatment plan), adjusting a final position of teeth (e.g., for a preexisting treatment plan), adding overcompensation to one or more specific teeth movements, applying a more conservative staging pattern (e.g., staging with spaces for problematic contacts for a preexisting treatment plan), adding elastics to aligners, adding bite ramps to aligners, adding a compliance indicator for aligners, adding attachments to one or more patient teeth, adjusting one or more attachments previously applied to patient teeth (e.g., for a preexisting treatment plan), adding or removing one or more temporary anchorage devices, re-bonding an attachment to a tooth, performing interproximal reduction, extracting a tooth, performing palatal expansion, adjusting a position of one or more teeth in a sagittal, vertical and/or transverse plane, rotating one or more teeth about one or more axis, increasing a separation between two or more teeth, changing a shape of an aligner, adding an implant, adding a restoration, (e.g., a veneer, a bridge, a crown, a cap, etc.), and so on. Treatment plan generator 276 may additionally or alternatively output a suggestion of one or more stages for a treatment plan, one or more recommendations for actions to be performed either at a start of treatment or at one or more stages of treatment, and so on.

In some embodiments, treatment plan generator 276 includes multiple machine learning models that were trained using reinforcement learning with different cost functions or reward functions. For example, one model may have been trained with a reward function that rewarded minimizing a number of stages for orthodontic treatment while still achieving target dentition, another model may have been trained with a reward function that rewarded minimizing an amount of time that orthodontic treatment is performed, and another model may have been trained with a reward function that rewarded a combination of minimized stages of treatment and minimized treatment time. Other models may have also been trained using reward functions that rewarded other goals. In embodiments, one or more goals for a treatment plan are provided to treatment plan generator 276, and treatment plan generator 276 selects a model that was trained using a reward function associated with the selected goal or goals. In some embodiments, a single machine learning model is trained in a manner that enables goals to be provided as an input to the model, and which generates an output that optimized for the input goals.

A doctor may implement the output treatment plan, or modify the treatment plan and then implement the modified treatment plan. In some embodiments, treatment plan generator 276 outputs multiple treatment plans, which may have different numbers of stages, different tooth movements within stages, different actions to perform at one or more stages, etc., and a doctor may select a treatment plan to use from the multiple treatment plans.

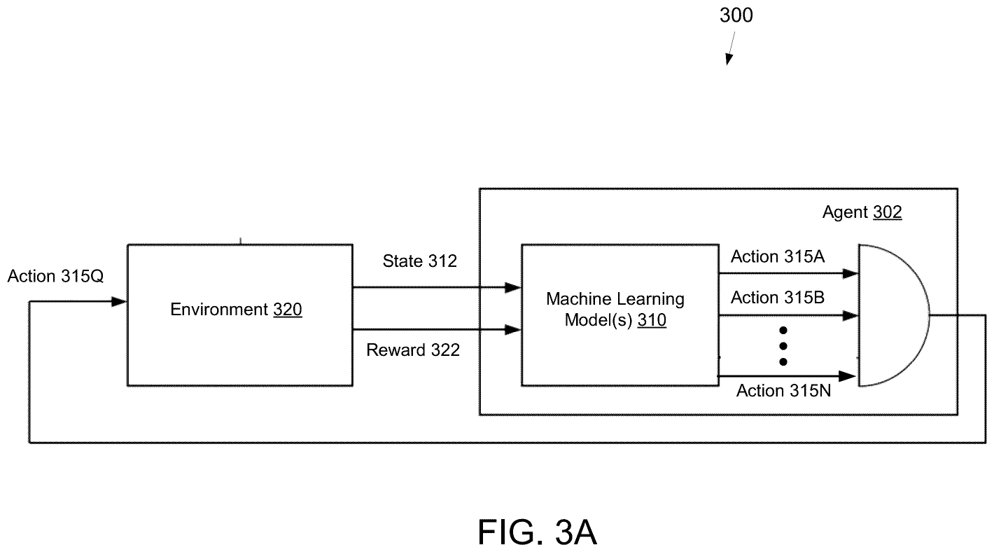

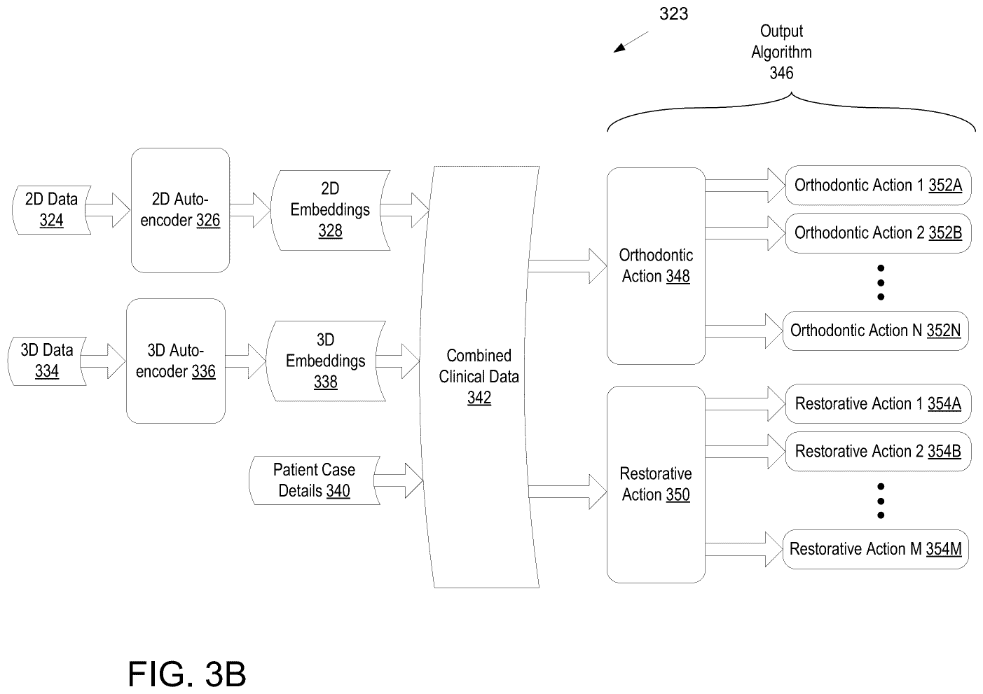

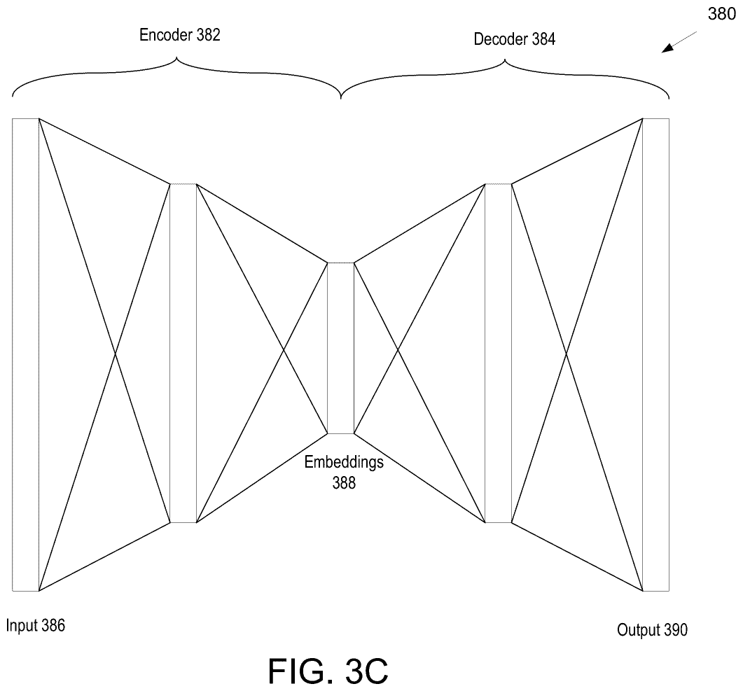

FIGS. 3A-12 below describe example applications of automated orthodontic treatment plan and/or ortho-restorative treatment plan generation and recommendation. Some examples are described with reference to flow charts describing processes of performing automatic orthodontic treatment plan generation and/or alteration. The flow charts provide example processes that may be performed by system 100 of FIG. 1 (e.g., by computing devices 105 and/or 106). The methods depicted in FIGS. 3-12 may be performed by a processing logic that may comprise hardware (e.g., circuitry, dedicated logic, programmable logic, microcode, etc.), software (e.g., instructions run on a processing device to perform hardware simulation), or a combination thereof.

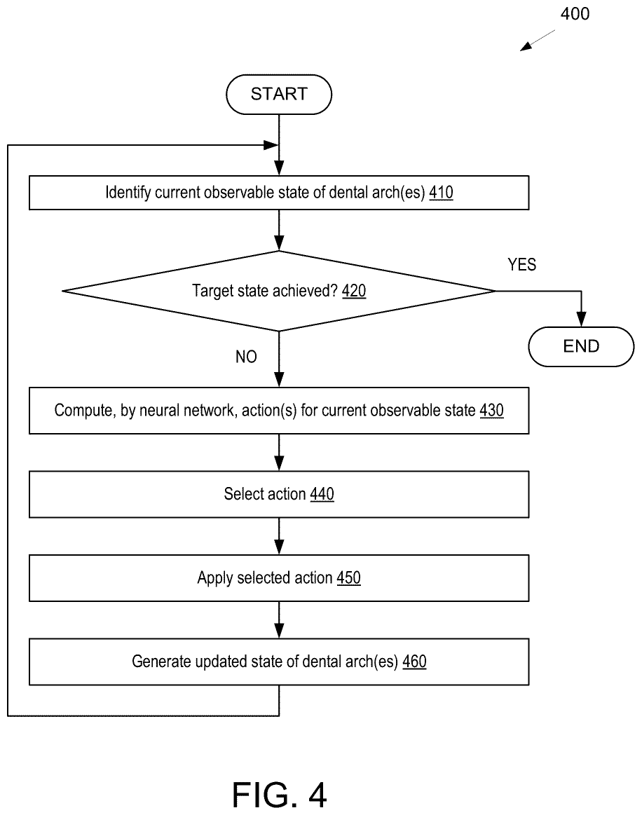

FIG. 3A illustrates application of reinforcement learning to train one or more machine learning models to generate orthodontic treatment plans and/or ortho-restorative treatment plans, in accordance with an embodiment of the present disclosure. In some implementations, treatment plan generator 276 or one or more machine learning models of treatment plan generator 276 of FIG. 2 may be trained by a reinforcement learning method, which causes the treatment plan generator 276 to select actions and/or stages of orthodontic treatment in order to maximize a cumulative reward over a treatment plan from a current observable state to a target observable state. Accordingly, a training session may involve running one or more agents 302, such that each agent 302 is assigned a certain goal (e.g., assigning certain values to one or more treatment conditions or treatment parameters, such as achieving a certain observable state of a patient's dentition or of a treatment plan within a target number of stages (e.g., with a minimal required number of stages) or a target amount of treatment time (e.g., with a minimal amount of treatment time)). Goals may include clinical goals and/or business goals in embodiments. In embodiments states are defined using a set of clinical measurements. In some embodiments, a state is defined as a combination of initial or current clinical measurements (e.g., an initial 3D model of a dental arch) and next clinical measurements (e.g., a 3D model of the dental arch after application of one or more actions). In some embodiments, a state is defined as a combination of initial or current clinical measurements (e.g., an initial 3D model of a dental arch) and a change in clinical measurements (e.g., a flow diagram indicating a magnitude and direction of change for locations on the 3D model). In some embodiments, clinical measurements are embedded in a learned lower-dimensional representation. For such embodiments, the machine learning model may generate a lower-dimensional representation for a current state and/or next state of a patient's dental arch(es) based on input clinical data. As shown in FIG. 4, which schematically illustrates operation of an agent 302 implemented in accordance with aspects of the present disclosure, the agent 302 may cause one or more trained machine learning (ML) models 310 (e.g., such as a neural network) 310 to iteratively generate stages of orthodontic treatment and/or actions to be performed at stages of orthodontic treatment. At every iteration, the agent 302 may feed, to one or more trained ML models 310, a vector of numeric values identifying an observable state 312. The observable state 312 may be represented, e.g., by a predicted state of a patient's dentition. Each action may correspond to a clinical approach to treatment, and may include adjustments to one or more teeth, addition of attachments, performance of IPR, widening an arch, proclining or inclining an incisor or other tooth, and/or any of the other actions described herein. Actions may include orthodontic actions, restorative dental actions, or a combination of orthodontic actions and restorative dental actions in embodiments.

Upon receiving a representation of the observable state 312, the one or more trained ML models 310 would process the observable state to produce a set of possible actions 315A-315N and their respective scores, such that a score associated with a particular action 315 indicates the likelihood of that an action triggering an observable state transition that belongs to the shortest path from the current observable state to a desired observable state (i.e., the shortest number of stages of orthodontic treatment needed to progress from a patient's current dentition to a patient's target dentition). In some embodiments, only orthodontic actions are output. In some embodiments, only restorative dental actions are output. In some embodiments, both orthodontic actions and restorative dental actions are output.

The agent 302 selects, with a known probability &, either a random action or the action 315 associated with the highest score among the candidate actions produced by the neural network. The action may be generation of a new stage of treatment and/or may include one or more actions for a doctor to perform in association with a stage of treatment, such as IPR, tooth extraction, or any of the other actions discussed herein. The probability & may be chosen as a monotonically-decreasing function of the number of training iterations, such that the probability & would be close to one at the initial iterations (thus forcing the agent to prefer random user interface actions over the actions produced by the untrained agent) and then would decrease with iterations to asymptotically approach a predetermined low value, thus giving more preference to the neural network output as the training progresses.

The agent 302 communicates the selected action 315Q to an environment 320. The environment 320 may represent a current state of a patient's dentition. Environment 320 applies the action 315Q to the current state of the patient's dentition, and returns a new observable state 312 and an optional reward 322 to the agent 302. In embodiments, an applied reinforcement learning technique incudes Q learning, deep Q learning networks, double deep Q learning networks, or another reinforcement learning technique.

The iterations may continue until a target observable state (e.g., a final target state of the patient's dentition) is reached or until an error condition is detected (e.g., a predetermined threshold number of stages of orthodontic treatment is exceeded or the neural network returning no valid actions for the current observable state).

During training of the ML model(s) 310 in reinforcement learning, actions may be taken from historical clinical data. Favorable actions (e.g. shorter than expected treatment times) may be rewarded, while unfavorable actions (e.g. longer treatment times) may be penalized. Model parameters are updated to make favorable actions for a given state more likely and unfavorable actions less likely.

Upon completing training, processing logic may validate the trained model by running it multiple times with added noise forcing the agent 302 to select, with a known small probability y, either a random action or the action associated with the highest score among the candidate actions produced by the one or more ML models 310. The validated models may be stored in the model storage 245 of FIG. 2. Once the system is trained, during inference, a selection action 315A-N may be an action having a highest output probability.

FIG. 3B illustrates an example machine learning model architecture 323, in accordance with an embodiment of the present disclosure. In one embodiment, the machine learning model architecture 323 corresponds to one or more machine learning models 310 of FIG. 3A. As shown in FIG. 3A, an agent 302 may receive information from environment 320 about a current state 312, which may be input into one or more ML models 310. The details of the machine learning architecture may differ greatly depending on the types of inputs in embodiments. Inputs may include patient case details 340 (e.g. age, gender, underlying health issues, etc.), 2D data 324 (e.g., 2D images of teeth, of patient smiles, etc.) and/or 3D data 334 (e.g., 3D intraoral scans, 3D models generated from intraoral scans, etc.). One method to combine these different data types includes specialized networks (e.g., auto-encoders) that convert the 2D data 324 and/or 3D data 334 into lower-dimensional embeddings (e.g., 2D embeddings 328 and/or 3D embeddings 338). As shown, a 2D auto-encoder 326 may convert 2D data 324 into 2D embeddings 328, and a 3D auto-encoder 338 may convert 3D data 334 into 3D embeddings 338 in embodiments. Embeddings may be lower-dimensional representations of input data. In some embodiments, the 2D embeddings and the 3D embeddings have a same dimensionality and/or data format. In one embodiment, one or more convolutional layers and/or transformer layers of a neural network (e.g., of a 2D auto-encoder 326) are used to generate 2D embeddings 328. In one embodiment, one or more layers of a graph neural network or 3D convolutional network are used to generate the 3D embeddings 338 from the 3D data 334.

In some embodiments, the 2D embeddings 328 and/or 3D embeddings 338 are combined with patient case details 340 to generate combined clinical data 342. The combined clinical data 342 may be fed into an output algorithm 346. The output algorithm may be another machine learning model (or additional machine learning model layers), which may constitute a neural network or another architecture such as a decision tree.

The output algorithm 346 (e.g., trained ML model) may output one or more classes or types of actions, such as orthodontic actions 348 and/or restorative actions 350. In embodiments, the output algorithm 346 may determine whether an orthodontic action 348 and/or a restorative action 350 is appropriate. Output algorithm 346 may then determine which among many possibilities for the determined type of action(s) is an appropriate action. For example, the output algorithm 346 may determine which of orthodontic action 1 352A, orthodontic action 2 352B through orthodontic action N 352N is an optimal orthodontic action to perform. Similarly, output algorithm 346 may determine which of restorative action 1 354A, restorative action 2 354B through restorative action N 354N is an optimal restorative action to perform. In embodiments, output actions may include probability values and/or confidence values.

Specialized 2D and/or 3D networks (e.g., 2D autoencoder 326 and/or 3D autoencoder 336) may be trained directly with other parameters during reinforcement learning. Alternatively, the 2D and/or 3D networks may be trained separately from the reinforcement learning. Training performed separately may performed be from a different, related task, such as tooth numbering for 2D images or 3D scans, for example. Training separately may also come from auto-encoder or similar unsupervised learning frameworks, which learn how to compress data with high fidelity.

FIG. 3C illustrates an example auto-encoder architecture 380, in accordance with an embodiment of the present disclosure. An autoencoder is a type of neural network designed to learn efficient codings (embeddings) of input data. As shown, the auto-encoder architecture 380 may include an encoder 382 and a decoder 384. Each of the encoder 382 and decoder 384 may include one or more layers. The encoder 382 is the part of the network that transforms input data into a compressed representation, referred to as a latent space or embedding 388. The encoder 382 reduces the dimensionality of the data while preserving essential features of the data. The decoder 384 generates an output 390, that is a reconstruction of the original input 386, from the embedding 388. The goal is for the output 390 to be as close as possible to the input 386. Once the autoencoder is fully trained, the decoder 384 may be discarded in some embodiments. For example, the 2D autoencoder 326 and/or 3D autoencoder 336 may omit a decoder in embodiments.

FIG. 4 depicts an example method of generating an orthodontic treatment plan or ortho-restorative treatment plan by a trainable agent implemented in accordance with one or more aspects of the present disclosure. As noted herein above, the trainable agents may be employed for generation of treatment plans, for generation of recommendations of actions for a doctor to perform for an orthodontic treatment plan, and so on. In some embodiments, the agent corresponds to treatment plan generator 276 of FIG. 2. In an illustrative example, generation of an optimal treatment plan may involve employing multiple trainable agents to achieve various target observable states (e.g., target dentition).

In embodiments, method 400 may be implemented by the treatment plan generator 276 of FIG. 2. Method 400 and/or each of its individual functions, routines, subroutines, or operations may be performed by one or more processors of a computing device (e.g., computing device 1300 of FIG. 13). In certain implementations, method 400 may be performed by a single processing thread. Alternatively, method 400 may be performed by two or more processing threads, each thread executing one or more individual functions, routines, subroutines, or operations of the method. In an illustrative example, the processing threads implementing method 400 may be synchronized (e.g., using semaphores, critical sections, and/or other thread synchronization mechanisms). Alternatively, the processing threads implementing method 400 may be executed asynchronously with respect to each other. Therefore, while FIG. 4 and the associated description lists the operations of method 400 in certain order, various implementations of the method may perform at least some of the described operations in parallel and/or in arbitrary selected orders.

As schematically illustrated by FIG. 4, at block 410, processing logic identifies a current observable state of a patient's dentition. The current observable state may be identified based on processing of clinical data, such as image data, scan data, a 3D model, etc. of the patient's dental arch(es). In some implementations, the current observable state of the patient's dentition may be represented by a vector of numeric values characterizing one or more parameters of the patient's dentition.

Responsive to determining, at block 420, that the current observable state matches the target observable state, the method terminates; otherwise, the processing continues at block 430.

At block 430, the computing device feeds the current observable state (e.g., a vector of numeric values representing the current observable state) to a neural network or other trained ML model, which may generate a plurality of actions (e.g., a plurality of different options for a next stage of orthodontic treatment and/or actions to be performed by a doctor to reach the next stage of orthodontic treatment) available at the current observable state and their respective action scores. The action scores may be represented by positive integer or real values.

At block 440, processing logic selects, based on the action scores, a next stage of orthodontic treatment or ortho-restorative treatment and/or one or more associated actions from the plurality of possible next stages of treatment. In an illustrative example, the computing device selects the next stage of orthodontic treatment associated with the optimal (e.g., maximal or minimal) score among the scores associated with the next stages of orthodontic treatment produced by the neural network. In another illustrative example, e.g., for training the neural network, processing logic selects, with a known probability &, either a random action or the action associated with the highest score among the user interface actions produced by the neural network, as described in more detail herein above.

At block 450, processing logic selects the selected next stage of orthodontic treatment and/or applies the selected action to the current observable state of the patient's dentition, as described in more detail herein above. At block 460, the next state of the patient's dentition (e.g., upper and/or lower dental arches) associated with the determined next stage of treatment is generated and becomes the new observable state of the patient's dentition.

The operations of block 410-460 are repeated iteratively until the target observable state of the patient's dentition is reached. Accordingly, responsive to completing operations of block 460, the method loops back to block 410. In some implementations, responsive to failing to achieve the desired observable state of the patient's dentition within a predefined number of iterations, the processing logic may initiate re-training of the neural network in order to modify one or more parameters of the neural network, as described in more detail herein above.

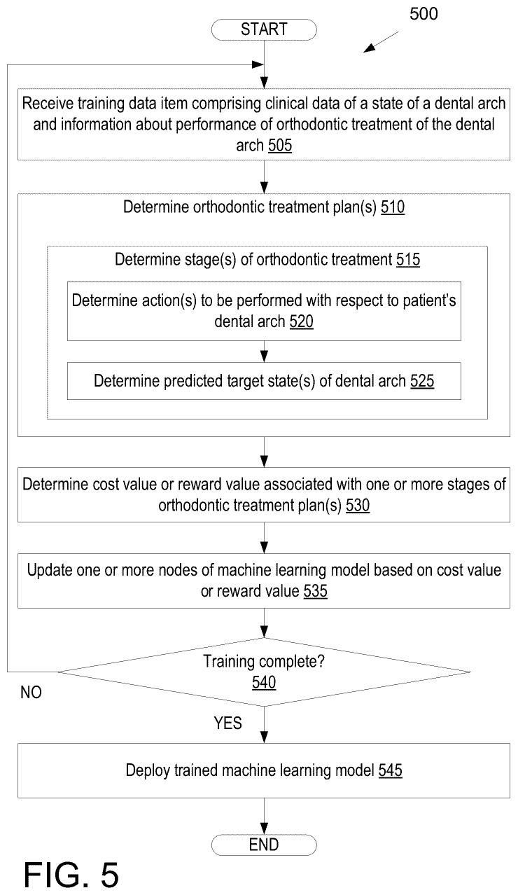

FIG. 5 illustrates a flow diagram for a method 500 of training a machine learning model to generate an orthodontic treatment plan or ortho-restorative treatment plan, in accordance with an embodiment. In some embodiments, the machine learning model corresponds to, or is included in, treatment plan generator 276 of FIG. 2. At block 505 of method 500, processing logic receives a training data item comprising clinical data of a state of a dental arch and information about performance of orthodontic treatment of the dental arch. At block 510, processing logic determines one or more orthodontic treatment plans for treatment of the dental arch. This may include at block 515 determining one or more stages of orthodontic treatment. Determining a stage of orthodontic treatment may include at block 520 determining one or more actions to be performed with respect to a patient's dental arch, such as moving one or more teeth in one or more directions by a determined amount, rotating one or more teeth about one or more axes by a determined amount, performing IPR, performing palatal expansion, performing tooth extraction, adding attachments to one or more teeth, removing attachments from one or more teeth, and so on. Actions may include actions to be performed by a doctor (e.g., such as IPR or tooth extraction) as well as actions that do not need to be performed by a doctor (e.g., such as designing an aligner to apply forces that move and/or rotate one or more teeth). Determining a stage of orthodontic treatment may additionally include determining a predicted target state of the upper and/or lower dental arch as a result of the actions. A treatment plan may include a sequence of treatment stages, each of which may include associated actions and an associated predicted state that will result from treatment at that stage of treatment.

At block 530, processing logic determines a cost value or reward value associated with the determined one or more stages of orthodontic treatment. The cost value or reward value may be computed by applying the determined actions, stages of treatment and/or states associated with the stages of treatment to a cost function or reward function. The cost/reward function may be tuned to reward and/or penalize particular outcomes. For example, the cost/reward function may be configured to reward treatment plans that achieve a target dentition with fewer stages of treatment and to penalize treatment plans that achieve the target dentition with a greater number of treatment stages. At block 535, processing logic updates one or more nodes of the machine learning model based on the cost value or reward value (e.g., based on back propagation).

At block 540, processing logic determines whether training is complete. If training is complete, the trained machine learning model may be deployed to a treatment planning application at block 545. If training is not complete, the method may return to block 505, and a new training data item may be received for further training of the machine learning model.

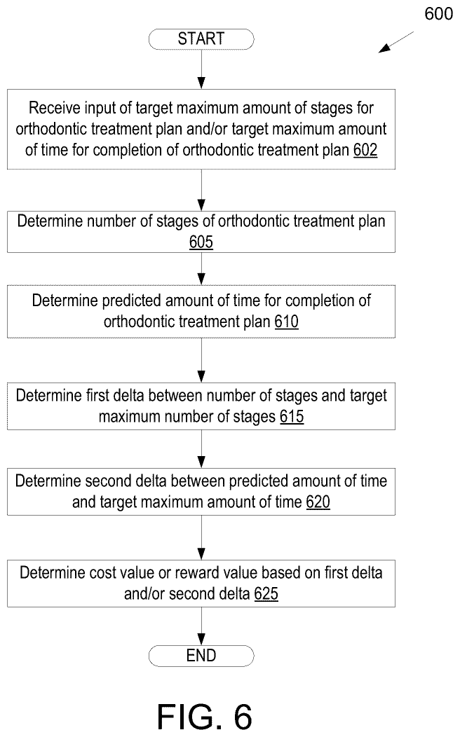

FIG. 6 illustrates a flow diagram for a method 600 of determining a cost value or reward value to apply for one or more stages of treatment of a generated treatment plan, in accordance with an embodiment. In some embodiments, method 600 is performed at block 530 of method 500. At block 602, processing logic may receive an input of a target maximum amount of stages for orthodontic treatment and/or for a target maximum amount of time for completion of orthodontic treatment. Additionally, or alternatively, processing logic may receive a goal to minimize a number of stages and/or to minimize an amount of time that treatment is performed. Alternatively, processing logic may determine one or more goals, and/or may apply a cost function or reward function that is already tailored for optimization of one or more predetermined goals.

At block 605, processing logic may determine a number of stages of a generated orthodontic treatment plan. At block 610, processing logic may determine a predicted amount of time for completion of the orthodontic treatment plan. At block 615, processing logic may determine a first delta between the number of stages and a target maximum number of stages. At block 620, processing logic may determine a second delta between the predicted amount of time and a target maximum amount of time. At block 625, processing logic may determine a cost value or reward value based on the first and/or second delta. The greater the delta, the lower the reward and/or the greater the cost in embodiments.

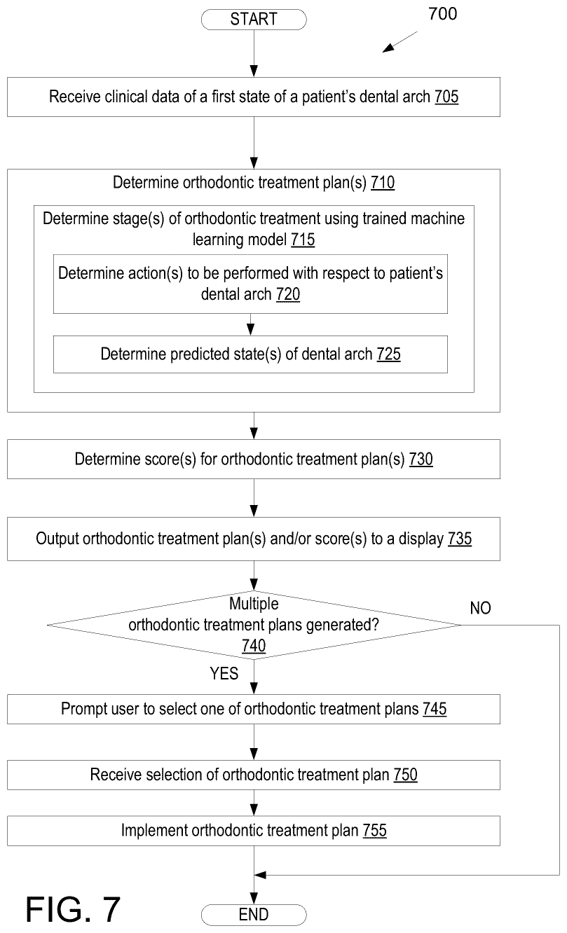

FIG. 7 illustrates a flow diagram for a method 700 of generating an orthodontic treatment plan or ortho-restorative treatment plan using a trained machine learning model, in accordance with an embodiment. At block 705 of method 700, processing logic receives clinical data of a first state of a patient's dental arch and/or of a target final sate of the patient's dental arch. At block 710, processing logic determines one or more orthodontic treatment plans and/or ortho-restorative treatment plans for treating the patient's dental arch (e.g., for correcting one or more malocclusions of the patient's dental arch) based on processing the clinical data. In one embodiment, this includes at block 715 determining one or more stages of orthodontic treatment and/or ortho-restorative treatment using a trained machine learning model (e.g., that may have been trained using reinforcement learning). Generating a stage of treatment may include determining one or more actions to be performed with respect to the patient's dental arch at block 720 and determining a predicted state of the patient's dental arch after application of the one or more actions. The predicted state of the patient's dentition (e.g., of an upper and/or lower dental arch) may include a generated 3D model of the patient's upper and/or lower dental arches in embodiments. The 3D model of the dental arches may be automatically generated by the machine learning model in embodiments. The actions may include movements, translations, rotations, etc. of one or more teeth, extraction of one or more teeth, IPR between one or more teeth, and so on. In some embodiments, processing logic determines a sequence of stages of orthodontic treatment and/or ortho-restorative treatment iteratively until a target state of the patient's dental arch is reached.

At block 730, processing logic determines scores for each of the one or more determined orthodontic treatment plans. At block 735, processing logic may outputs the one or more generated treatment plans and their associated scores to a display.

In one embodiment, at block 740 processing logic determines if multiple treatment plans were generated. If multiple treatment plans were generated, then at block 745 a user may be prompted to select one of the orthodontic treatment plans. The multiple treatment plans may include one or more ortho-restorative treatment plans that include restorative options such as allowable dimensions for use of veneers of different types and thickness combined allowing for minimal or reasonable removal of enamel and tooth mass. Some treatment plans allow for larger dental movements to take place, hence reducing the need for further removal of tooth mass. Some treatment plans allow for additional steps in treatment with consideration that a target dental movement was not completed as planned in a previous stage of treatment. More than one option may exist at this point in treatment. One possibility is updating the treatment plan. Another possibility is changing a dental appliance (e.g., orthodontia aligner) to achieve another movement. Another ortho-restorative treatment plan example is the need for space to place a dental implant, where space is impinged upon by the roots of the adjacent teeth to the space at which the dental implant is to be placed. It can be necessary in such an instance to spread the roots of the adjacent teeth apart to allow for placement of the implant. In another example, an ortho-restorative treatment plan may call for insertion of a particular type of implant at an intermediate stage of treatment (e.g., after teeth have been moved to form a space to install the implant). However, not enough space may have been created for the implant at the stage of treatment at which the implant was to be installed. In response, processing logic may add additional treatment stages to allow more time for increasing the space where the implant is to be inserted, or may update the treatment plan to use a different type of implant that requires less space. In some embodiments, deviant ortho-restorative treatment plans may be generated, one for each of the possible options.

At block 750, processing logic may then receive selection of one of the automatically generated treatment plans. Alternatively, processing logic may automatically select an orthodontic treatment plan or ortho-restorative treatment plan associated with a highest score (e.g., which may correlate to a highest reward or lowest cost). At block 755, the selected orthodontic treatment plan may be implemented.

Method 700 may be implemented both before orthodontic treatment is begun and at any intermediate state during orthodontic treatment. A patient's teeth may not respond to treatment as predicted. For example, teeth may move more quickly or more slowly than anticipated. If method 700 is implemented during an intermediate stage of treatment, then one or more prior states of the patient's dentition may be input into the machine learning model along with the current state of the machine learning model. In some embodiments, a previously generated treatment plan is stored, and/or a current state of implementation of the previously generated treatment plan is stored. The previously generated treatment plan and/or current state of implementation of the approved treatment plan may be input into the machine learning model in embodiments. The machine learning model may output an updated treatment plan that may replace one or more remaining stages of the previously generated treatment plan.

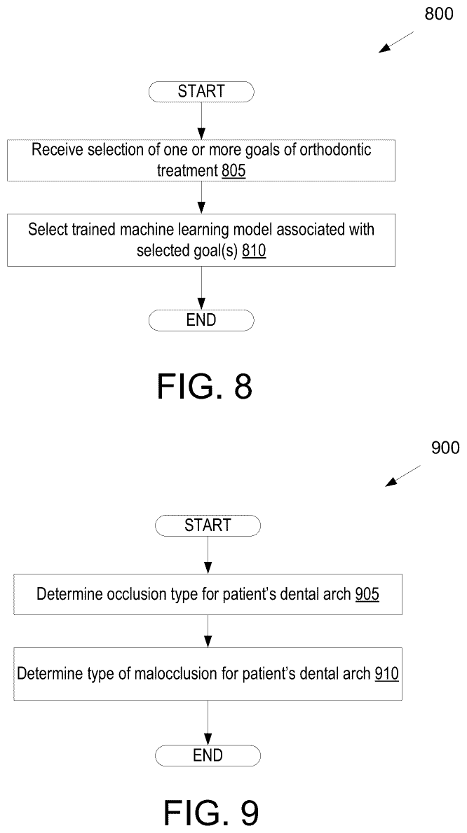

FIG. 8 illustrates a flow diagram for a method 800 of selecting a trained machine learning model to generation of an orthodontic treatment plan, in accordance with an embodiment. At block 805 of method 800, processing logic receives a selection of one or more goals for orthodontic treatment, such as a goal to minimize a number of stages and/or a goal to minimize an amount of treatment time. At block 810, processing logic may select a trained machine learning model associated with the selected goal or goals. This machine learning model is the machine learning model that may be applied at method 700 in embodiments.

FIG. 9 illustrates a flow diagram for a method 900 of determining information usable to assist in determination of actions for one or more stages of an orthodontic treatment plan, in accordance with an embodiment. In one embodiment, method 900 is executed using a trained machine learning model that is trained to receive an input of clinical data (e.g., intraoral scans, 3D models, 2D images, etc.), and to output one or more classifications about a patient's dentition. At block 905, clinical data is input into the trained machine learning model, and the machine learning model outputs an occlusion type for the patient's dental arch. The clinical data may include a current state of the patient's dental arches (e.g., a 3D model of the patient's dental arches as they currently exist), and/or a predicted future state of the patient's dental arch associated with a stage of treatment (e.g., a synthetically generated 3D model of the upper and lower dental arch). The occlusion type may include, for example, an open bite, a cross bite, an overbite, an underbite, a deep bite, and so on.

At block 910, clinical data is input into the trained machine learning model, and the machine learning model outputs one or more classes of malocclusion, such as a class I malocclusion, a class Il malocclusion, a class Ill malocclusion, and so on. Additionally, or alternatively, the trained machine learning model may output a current bite relation between an upper and lower dental arch. The clinical data may include a current state of the patient's dental arches (e.g., a 3D model of the patient's dental arches as they currently exist, including a current bite relation between the upper and lower dental arches), and/or a predicted future state of the patient's dental arch associated with a stage of treatment (e.g., a synthetically generated 3D model of the upper and lower dental arch, and optionally a determined bite relationship between the upper and lower dental arch). In some embodiments, the operations of block 905 and 910 are performed together.

Method 900 may be performed, for example, at block 710 of method 700 in embodiments.

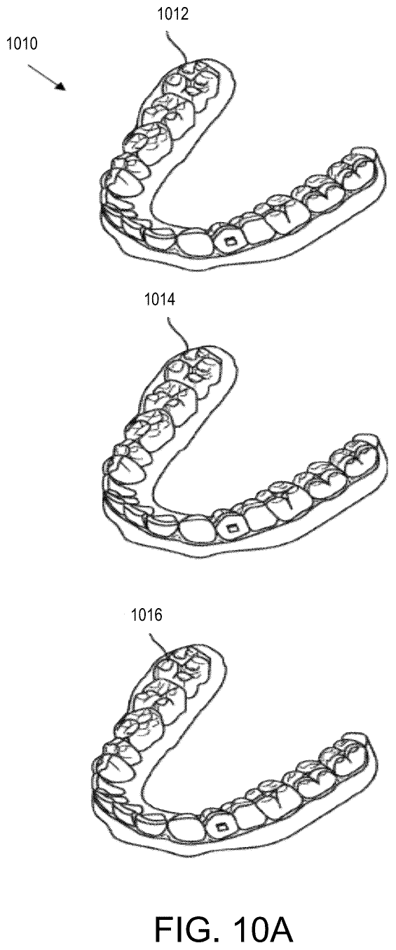

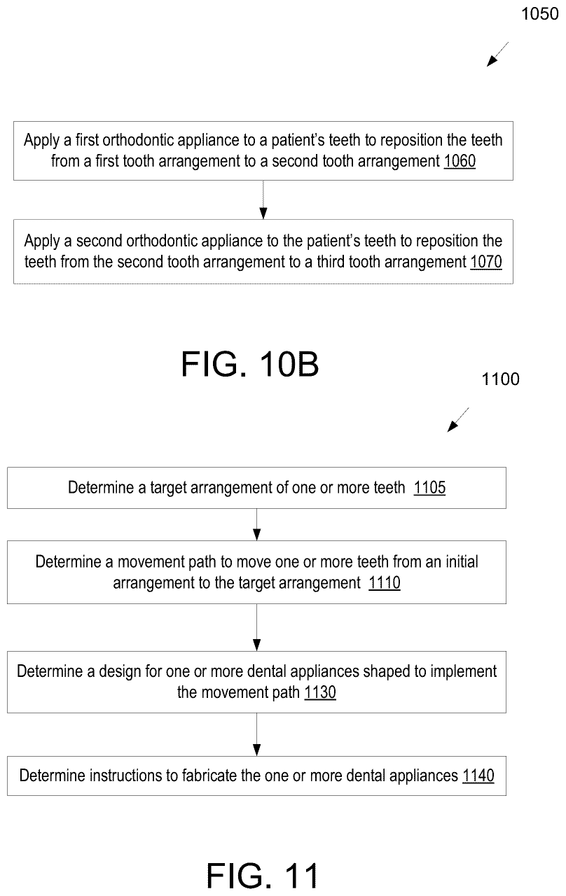

FIG. 10A illustrates a tooth repositioning system 1010 including a plurality of appliances 1012, 1014, 1016. The appliances 1012, 1014, 1016 can be designed based on generation of a sequence of 3D models of dental arches, which may be generated according to the techniques discussed herein above. Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1010 can include a first appliance 1012 corresponding to an initial tooth arrangement, one or more intermediate appliances 1014 corresponding to one or more intermediate arrangements, and a final appliance 1016 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 1012, 1014, 1016 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 1012, 1014, 1016 after the digital models of the appliances 1012, 1014, 1016 have been processed by processing logic of a computing device, such as the computing device in FIG. 13. The processing logic may include hardware (e.g., circuitry, dedicated logic, programmable logic, microcode, etc.), software (e.g., instructions executed by a processing device), firmware, or a combination thereof. For example, one or more operations may be performed by a processing device executing treatment plan generator 276 of FIG. 2.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. In one embodiment, treatment plan generator 276 outputs a desired treatment outcome based on processing the virtual 3D model of the patient's dental arch (or other dental arch data associated with the virtual 3D model). Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. In one embodiment, for each treatment stage treatment plan generator 276 outputs a different 3D model of the dental arch. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 1012, 1014, 1016 can be pressure formed or thermoformed over the molds. Each mold may be used to fabricate an appliance that will apply forces to the patient's teeth at a particular stage of the orthodontic treatment. The appliances 1012, 1014, 1016 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments. In embodiments, the additional information may be automatically added to a generated 3D model by treatment plan generator 276 in generation of the 3D model.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. In one embodiment, treatment plan generator 276 outputs a cutline for an appliance associated with a 3D model output by the dental arch generator 268. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 10B illustrates a method 1050 of orthodontic treatment using a plurality of appliances, in accordance with embodiments. The method 1050 can be practiced using any of the appliances or appliance sets described herein. In block 1060, a first orthodontic appliance is applied to a patient's teeth in order to reposition the teeth from a first tooth arrangement to a second tooth arrangement. In block 1070, a second orthodontic appliance is applied to the patient's teeth in order to reposition the teeth from the second tooth arrangement to a third tooth arrangement. The method 1050 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 11 illustrates a method 1100 for designing an orthodontic appliance to be produced by direct or indirect fabrication, in accordance with embodiments. The method 1100 can be applied to any embodiment of the orthodontic appliances described herein, and may be performed using one or more trained machine learning models in embodiments. Some or all of the blocks of the method 1100 can be performed by any suitable data processing system or device, e.g., one or more processors configured with suitable instructions.

At block 1105 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model such as treatment plan generator 276 of FIG. 2. With a specification of the desired final positions of the teeth and a digital representation of the teeth themselves, the final position and surface geometry of each tooth can be specified to form a complete model of the tooth arrangement at the desired end of treatment.

In block 1110, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, movement paths are determined by a trained machine learning model such as treatment plan generator 276. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. In one embodiment, the force system is determined by a trained machine learning model such as data arch generator 268. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 1130, a design for one or more dental appliances shaped to implement the movement path is determined. In one embodiment, the one or more dental appliances are shaped to move the one or more teeth toward corresponding incremental arrangements. In one embodiment the orthodontic application is determined by treatment plan generator 276. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

In block 1140, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

In one embodiment, instructions for fabrication of the one or more dental appliances are generated by treatment plan generator 276. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 1100 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 1100 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 1100 may be performed with any suitable fabrication system or device, such as the embodiments described herein. Some of the blocks may be optional, and the order of the blocks can be varied as desired.

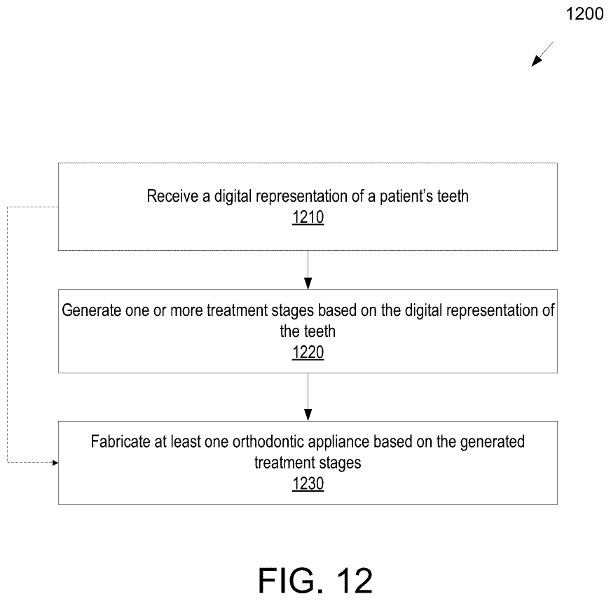

FIG. 12 illustrates a method 1200 for digitally planning an orthodontic treatment and/or design or fabrication of an appliance, in accordance with embodiments. The method 1200 can be applied to any of the treatment procedures described herein and can be performed by any suitable data processing system.

In block 1210, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1220, one or more treatment stages are generated based on the digital representation of the teeth. In some embodiments, the one or more treatment stages are generated based on processing of input dental arch data by a trained machine learning model such as treatment plan generator 276. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1230, at least one orthodontic appliance is fabricated based on the generated treatment stages. For example, a set of appliances can be fabricated, each shaped according a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 12, design and/or fabrication of an orthodontic appliance, and perhaps a particular orthodontic treatment, may include use of a representation of the patient's teeth (e.g., receive a digital representation of the patient's teeth at block 1210), followed by design and/or fabrication of an orthodontic appliance based on a representation of the patient's teeth in the arrangement represented by the received representation.

FIG. 13 illustrates a diagrammatic representation of a machine in the example form of a computing device 1300 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computer device 1300 corresponds to computing devices 105 of FIG. 1.

The example computing device 1300 includes a processing device 1302, a main memory 1304 (e.g., read-only memory (ROM), flash memory, dynamic random access memory (DRAM) such as synchronous DRAM (SDRAM), etc.), a static memory 1306 (e.g., flash memory, static random access memory (SRAM), etc.), and a secondary memory (e.g., a data storage device 1328), which communicate with each other via a bus 1308.

Processing device 1302 represents one or more general-purpose processors such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1302 may also be one or more special-purpose processing devices such as an application specific integrated circuit (ASIC), a field programmable gate array (FPGA), a digital signal processor (DSP), network processor, or the like. Processing device 1302 is configured to execute the processing logic (instructions 1326) for performing operations and steps discussed herein.

The computing device 1300 may further include a network interface device 1322 for communicating with a network 1364. The computing device 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse), and a signal generation device 1320 (e.g., a speaker).

The data storage device 1328 may include a machine-readable storage medium (or more specifically a non-transitory computer-readable storage medium) 1324 on which is stored one or more sets of instructions 1326 embodying any one or more of the methodologies or functions described herein, such as instructions for a treatment planning application 1350. A non-transitory storage medium refers to a storage medium other than a carrier wave. The instructions 1326 may also reside, completely or at least partially, within the main memory 1304 and/or within the processing device 1302 during execution thereof by the computer device 1300, the main memory 1304 and the processing device 1302 also constituting computer-readable storage media.

The computer-readable storage medium 1324 may also be used to store treatment planning application 1350, which may correspond to the similarly named component of FIG. 1. The computer readable storage medium 1324 may also store a software library containing methods for treatment planning application 1350. While the computer-readable storage medium 1324 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present invention. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present invention have been described with reference to specific example embodiments, it will be recognized that the invention is not limited to the embodiments described, but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

1. A system comprising:
a computing device comprising a memory and one or more processing devices, wherein the computing device is configured to:
receive clinical data of a first state of one or more dental arches of a patient; and
determine one or more stages of an orthodontic treatment plan for correcting the one or more dental arches based on processing of the clinical data, wherein determining a stage of the orthodontic treatment plan comprises:
determining one or more actions to be performed with respect to the one or more dental arches; and
determining a target state of the one or more dental arches that is predicted to result at least in part from the one or more actions.

2. The system of claim 1, wherein the computing device is further configured to:
determine the orthodontic treatment plan comprising the one or more stages;
determine one or more additional orthodontic treatment plans;
output the orthodontic treatment plan and the one or more additional orthodontic treatment plans to a display;
receive a selection of the orthodontic treatment plan; and
implement the selected orthodontic treatment plan.

3. The system of claim 2, wherein the computing device is further configured to:
determine, for the orthodontic treatment plan, a first score associated with predicted accomplishment of one or more target conditions by the orthodontic treatment plan; and
determine, for each additional orthodontic treatment plan of the one or more additional orthodontic treatment plans, an additional score associated with accomplishment of the one or more target conditions by the additional orthodontic treatment plan.

4. The system of claim 3, wherein the first score and the additional scores of the one or more additional orthodontic treatment plans are output to the display.

5. The system of claim 1, wherein the processing of the clinical data is performed using a trained machine learning model, wherein the trained machine learning model outputs the one or more actions and the target state of the one or more dental arches.

6. The system of claim 5, wherein the first state of the one or more dental arches is an intermediate state achieved by a previously determined orthodontic treatment plan, and wherein the computing device is further configured to:
receive information associated with the previously determined orthodontic treatment plan;
determine a cost value or reward value based on a degree of similarity between the first state of the one or more dental arches and a predicted state of the one or more dental arches from the previously determined orthodontic treatment plan; and
update a training of the trained machine learning model based on the cost value or reward value, wherein the processing of the clinical data using the trained machine learning model is performed after updating the training of the trained machine learning model.

7. The system of claim 1, wherein the receiving of the clinical data and the determining of the one or more stages of the orthodontic treatment plan is performed prior to beginning orthodontic treatment of the one or more dental arches, and wherein the computing device is further configured to:
receive new clinical data of a new state of the one or more dental arches during an intermediate stage of the orthodontic treatment plan; and
determine, with or without user input, one or more updated stages of the orthodontic treatment plan based on processing of the new clinical data.

8. The system of claim 7, wherein the one or more updated stages comprise a final stage having a new target state of the one or more dental arches.

9. The system of claim 7, wherein the computing device is further configured to:
add one or more new intermediate stages to the orthodontic treatment plan in view of the new clinical data.

10. The system of claim 1, wherein the clinical data comprises at least one a color two-dimensional (2D) image of the one or more dental arches, a three-dimensional (3D) model of each of the one or more dental arches, an intraoral scan of the one or more dental arches, or an x-ray image of the one or more dental arches.

11. The system of claim 1, wherein the one or more actions comprise at least one of widening the one or more dental arches, adding one or more attachments to the one or more dental arches, extracting one or more teeth from the one or more dental arches, performing interproximal reduction for one or more teeth of the one or more dental arches, or securing the one or more dental arches to each other using elastics.

12. The system of claim 1, wherein the one or more actions comprise adjusting at least one of a position or an orientation of one or more teeth on the one or more dental arches.

13. The system of claim 1, wherein the one or more actions and the target state that are determined minimize at least one of a number of stages of the orthodontic treatment plan or a duration of orthodontic treatment performed according to the orthodontic treatment plan.

14. The system of claim 1, wherein the computing device is further configured to:
determine an occlusion type for the one or more dental arches based on processing of the clinical data, wherein the occlusion type comprises at least one of an open bite, a cross bite, or a deep bite;
wherein the one or more actions are determined based at least in part on the occlusion type.

15. The system of claim 1, wherein the computing device is further configured to:
determine a type of malocclusion for the one or more dental arches based on processing of the clinical data;
wherein the one or more actions are determined based at least in part on the type of malocclusion.

16. The system of claim 1, wherein the one or more dental arches comprise an upper dental arch of the patient, and wherein the computing device is further configured to:
receive clinical data of a first state of a lower dental arch of the patient; and
determine one or more stages of an orthodontic treatment plan for correcting the lower dental arch based on processing of the clinical data, wherein determining a stage of the orthodontic treatment plan for correcting the lower dental arch comprises:
determining one or more additional actions to be performed with respect to the lower dental arch; and
determining a target state of the lower dental arch that is predicted to result at least in part from the one or more additional actions.

17. The system of claim 1, wherein the computing device is further configured to:
determining a current relation between an upper dental arch of the one or more dental arches and a lower dental arch of the one or more dental arches, wherein determining the target state of the one or more dental arches that is predicted to result at least in part from the one or more actions comprises determining a target relation between the upper dental arch and the lower dental arch.

18. The system of claim 1, wherein determining the one or more actions to be performed with respect to the one or more dental arches comprises determining one or more restorative dental actions to be performed, the one or more restorative dental actions associated with one or more restorative options.

19. The system of claim 18, wherein:
the one or more restorative options comprise at least one of a) one or more allowable dimensions for use of a veneer, b) a type of veneer to use, or c) a veneer thickness; and
the one or more restorative dental actions to be performed comprise an amount of tooth mass to remove from a tooth that is to receive the veneer.

20. The system of claim 18, wherein a space to place a dental implant is impinged upon by roots of one or more teeth that are adjacent to the space, and wherein the one or more restorative dental actions to be performed comprise spreading the roots of the one or more teeth apart to allow for placement of the dental implant in the space.

21. The system of claim 1, wherein the computing device is further configured to:
cause one or more orthodontic aligners to be manufactured in accordance with the one or more stages of the orthodontic treatment plan.

22. The system of claim 1, further comprising:
an additional computing device configured to send the clinical data to the computing device; and
a storage device configured to store the orthodontic treatment plan.

23. A computer readable medium comprising instructions that, when executed by one or more processing devices, cause the one or more processing devices to perform operations comprising:
receiving, by the one or more processing devices, clinical data of a first state of one or more dental arches of a patient; and
determining, by the one or more processing devices, one or more stages of an orthodontic treatment plan for correcting the one or more dental arches based on processing of the clinical data, wherein determining a stage of the orthodontic treatment plan comprises:
determining one or more actions to be performed with respect to the one or more dental arches; and
determining a target state of the one or more dental arches that is predicted to result at least in part from the one or more actions.

24. A method comprising:
receiving, by one or more processing devices, clinical data of a first state of one or more dental arches of a patient; and
determining, by the one or more processing devices, one or more stages of an orthodontic treatment plan for correcting the one or more dental arches based on processing of the clinical data, wherein determining a stage of the orthodontic treatment plan comprises:
determining one or more actions to be performed with respect to the one or more dental arches; and
determining a target state of the one or more dental arches that is predicted to result at least in part from the one or more actions.
